(19) United States
(12) Patent Application Publication
(10) Pub. No.: US 2026/0306402 A1
(43) Pub. Date: Oct. 8, 2026

(54) AQUEOUS FORMULATIONS COMPRISING FULLERENE, STRUCTURED WATER, DISSOLVED HYDROGEN GAS, MINERALS AND ADDITIVES

(71) Applicant: BEST PLANET SCIENCE LLC, Wilmington, DE (US)

(72) Inventor: Tatiana Svetlana LEON CAMACHO, Bogotá (CO)

(21) Appl. No.: 19/480,667

(22) PCT Filed: May 3, 2024
(86) PCT No.: PCT/US2024/027717

§ 371 Date: Oct. 31, 2025

Related U.S. Application Data
(60) Provisional application No. 63/463,874, filed on May 3, 2023.

(51) Int. Cl. A61K 9/107; A61K 9/00; A61K 33/00; A61K 41/00; A61K 45/06; A61K 47/02; A61P 35/00

(52) U.S. Cl. CPC A61K 9/1075; A61K 9/0053; A61K 33/00; A61K 41/0057; A61K 45/06; A61K 47/02; A61P 35/00

(57) ABSTRACT

Provided is an aqueous formulation including fullerene, structured water having a three-dimensional helical structure of polygonal water molecules having a hollow lumen, dissolved hydrogen gas, minerals, and additives. The formulation provides a stable and biocompatible fullerene, which can be surrounded by micelles. The aqueous formulation has long-term stability, whereby the concentration of dissolved hydrogen gas in the aqueous formulation is maintained over time. Also provided are methods of treating cancer that include administering to a subject in need thereof an aqueous formulation including fullerene, structured water having a three-dimensional helical structure of polygonal water molecules having a hollow lumen, dissolved hydrogen gas, minerals, and additives.

### CROSS-REFERENCE TO RELATED APPLICATIONS

The present application claims priority to and the benefit of U.S. Provisional Application Ser. No. 63/463,874, titled AQUEOUS FORMULATIONS COMPRISING FULLERENE, STRUCTURED WATER, DISSOLVED HYDROGEN GAS, MINERALS AND ADDITIVES, filed May 3, 2023, the entire contents of which are incorporated by reference in their entirety, where permitted.

### TECHNICAL FIELD

The present application is directed to aqueous formulations that include fullerene in combination with structured water containing dissolved hydrogen gas, minerals and additives. The structured water has a three-dimensional helical structure of polygonal water molecules having a hollow lumen. The present application also is directed to methods for preparing the aqueous formulations. The aqueous formulations of the present application exhibit long-term stability, whereby the concentration of dissolved hydrogen gas in the aqueous formulation is maintained over time. The present application also is directed to aqueous formulations that include fullerene in combination with structured water containing dissolved hydrogen gas, minerals and additives for the treatment of cancer.

### BACKGROUND

The background description provided herein is for the purpose of generally presenting context of the disclosure. Unless otherwise indicated herein, the materials described in this section are not prior art to the claims in this application and are not admitted to be prior art, or suggestions of the prior art, by inclusion in this section.

Carbon nanoparticles, such as fullerenes and carbon nanotubes, have attracted attention as possible carriers for drugs (Lisik et al., International Journal of Molecular Sciences 2021, 22, 8341, 15 pages). Fullerenes have a unique structure and have demonstrated properties ideally suited for interaction with components in the cellular environment.

The high hydrophobicity of these molecules makes it difficult to solubilize them in water, and they tend to form aggregates. To address this, the molecules typically are functionalized. One of the most common ways to make fullerenes water-soluble is by adding hydrophilic groups to the surface of the molecule. These groups can be carboxylic acids, alcohols, amines or sulfonates, and are attached to the surface of the fullerene via covalent or non-covalent bonds. Hydrophilic groups allow the fullerenes to disperse or dissolve in aqueous solution. Another way to make fullerenes water-soluble is by forming complexes with water-soluble polymers. These polymers can be polyethylene glycol (PEG), dextrans, or polysaccharides, and are attached to the fullerene surface by non-covalent bonds. Complexes formed with water-soluble polymers are stable in solution and allow the fullerenes to dissolve and disperse in water. Water-soluble fullerenes have diverse applications, such as diagnostic and therapeutic agents in medicine, coating materials and additives in the materials industry, and in electronics and nanotechnology. In addition, water solubility also facilitates the manipulation and study of these molecules in solution.

While such functionalization can help to solubilize carbon nanoparticles, such as fullerenes, in aqueous solutions, the functionalization can modify the chemical and physical properties of the fullerene. For example, it was found the unmodified C60 fullerenes yield singlet oxygen upon irradiation more efficiently than water-soluble derivatives (Rathmore et al., J Chem Res. 2(2): 240-248 (2010)). This attribute makes the unmodified fullerene more desirable for some applications, such as photodynamic therapy. Modified fullerenes also showed a reduced ability to absorb free radicals compared to unmodified fullerene molecules. Accordingly, a need exists to provide dispersed fullerene molecules in an aqueous solution where the fullerenes exhibit increased stability and biocompatibility.

### SUMMARY

Provided herein are aqueous of solutions that address the above-mentioned problems associated with aqueous dispersions or solutions containing fullerenes.

Provided herein is an aqueous composition that includes a fullerene; a micelle-forming agent; a structured water having a three-dimensional helical structure of polygonal water molecules having a hollow lumen; a dissolved hydrogen gas; and minerals. The fullerene can include an unmodified C60 or C70 fullerene. The fullerene can include a C60 or C70 fullerene modified to include one or more than one hydrophilic group on a surface of the fullerene. The hydrophilic group can be a hydroxyl group, a carboxy group, a carboxylic acid group, an alcohol group, an amine group, a sulfonate group, or any combination thereof. The fullerene can be a polyhydroxylated fullerene, known as a fullerenol. The fullerene can include a C60 or C70 fullerene modified to be complexed with a water-soluble polymer. The water-soluble polymer can be selected from the group consisting of a polyethylene glycol, a dextran, a polyvinyl alcohol, a polysaccharide, and a protein. The amount of the fullerene in the aqueous composition can be an amount from about 1 mg/L to 30,000 mg/L.

In the aqueous compositions provided herein, the micelle-forming agent can be or include a surfactant, a liposoluble agent, an oil, or a combination thereof. The oil can be an essential oil, an olive oil, a coconut oil, or a combination thereof. The micelle-forming agent can be or include a nonionic surfactant. The nonionic surfactant can be selected from the group consisting of a sorbitan ester, a polyalkoxylated sorbitan fatty acid ester, a polyoxyethylene ether, a polysorbate, and a combination thereof. The polysorbate can be selected from the group consisting of polysorbate 20, polysorbate 40, polysorbate 60, and polysorbate 80. The amount of the micelle-forming agent can be 50 mg/L to 800 g/L.

In the aqueous compositions provided herein, the amount of hydrogen gas present can be in a range of 1 to 50 ppm. In the aqueous compositions provided herein, the minerals can be selected from the group consisting of calcium (Ca), magnesium (Mg), iron (Fe), zinc (Zn), copper (Cu), and selenium (Se). The minerals can be present in an amount from 0.005 mg/L to 800 mg/L.

The aqueous compositions provided herein can further include a sweetener. The sweetener can be selected from the group consisting of sucrose, stevia, steviol glycosides, monk fruit extract, sucralose, isomalt, aspartame, saccharin, acesulfame potassium, neotame, advantame, lactitol, xylitol, high-fructose corn syrup, and combinations thereof. The aqueous compositions provided herein can further include a flavoring agent. The aqueous compositions provided herein can further include a flavor enhancer selected from among a citric acid, a lactic acid, a sodium gluconate, a sodium gluconolactate, an ethyl lactate, a sodium lactate, a sodium acid sulfate, malic acid, and combinations thereof. In the aqueous compositions provided herein, the sweetener and/or the flavoring agent and/or the flavor enhancer, when present, can be present in an amount from about 0.0001 to 20 wt %.

Provided is an aqueous composition including a surfactant in an amount of 1000 mg/L; a flavor in an amount of 1 to 1500 mg/L; a sweetener in an amount of 1 to 100 mg/L; an unmodified C60 fullerene in an amount of 20 mg/L, 40 mg/L, 60 mg/L, or 80 mg/L; a Ca salt providing Ca in an amount of 586 mg/L; a Mg salt providing Mg in an amount of 325 mg/L; a Fe salt providing Fe in an amount of 5 mg/L; a Zn salt providing Zn in an amount of 5 mg/L; a Se salt providing Se in an amount of 0.01 mg/L; a Cu salt providing Cu in an amount of 0.2 mg/L; and hydrogen (H₂) in an amount of 20 ppm.

The surfactant can be polysorbate 80; the flavor can be benzaldehyde and can be present in an amount of 1.5 mg/L; and the sweetener can be stevia and can be present in an mount from 30 to 100 mg/L.

Also provided is an aqueous composition including a surfactant in an amount of 1000 mg/L; a flavor in an amount of 1 to 1500 mg/L; a sweetener in an amount of 1 to 100 mg/L; fullerenol in an amount of 200 mg/L, 400 mg/L, 600 mg/L, or 800 mg/L; a Ca salt providing Ca in an amount of 586 mg/L; a Mg salt providing Mg in an amount of 325 mg/L; a Fe salt providing Fe in an amount of 5 mg/L; a Zn salt providing Zn in an amount of 5 mg/L; a Se salt providing Se in an amount of 0.01 mg/L; a Cu salt providing Cu in an amount of 0.2 mg/L; and hydrogen (H₂) in an amount of 20 ppm. The surfactant can be polysorbate 80; the fullerenol can be C60 fullerenol; the flavor can be benzaldehyde and can be present in an amount of 1.5 mg/L; and the sweetener can be stevia and can be present in an mount from 30 to 100 mg/L.

Also provided is a method of administering photodynamic therapy to a subject in need thereof, the method comprising selecting a site of the subject where the photodynamic therapy is to be administered; administering to the subject an aqueous composition provided herein, which can be administered alone or in combination with a pharmaceutically acceptable carrier; and exposing the site to natural or artificial light for a period of time effective to achieve a therapeutic effect. The subject can have a disorder selected from among a cancer, psoriasis, acne, actinic keratosis, eczema, seborrhea and hyperkeratinosis. The subject can have a cancer selected from among breast, prostate, liver, colorectal, gastrointestinal, pancreatic, skin, thyroid, cervical, lymphoid, hematopoietic, bladder, lung, renal, ovarian, uterine, and head or neck cancer.

Also provided is a method of treating cancer in a subject, comprising administering to the subject a therapeutically effective amount of an aqueous composition provided herein, which can be administered alone or in combination with a pharmaceutically acceptable carrier. The cancer can be selected from among breast, prostate, liver, colorectal, gastrointestinal, pancreatic, skin, thyroid, cervical, lymphoid, hematopoietic, bladder, lung, renal, ovarian, uterine, and head or neck cancer. The cancer can be selected from among an adenocarcinoma, a medulloblastoma, chronic lymphocytic leukemia, and a cerebellar tumor. The aqueous composition provided herein can be administered alone or in combination with another therapeutic treatment, such as a chemotherapy, a radiotherapy, an immunotherapy, or a combination thereof.

The aqueous composition provided herein can be administered daily, or twice weekly, or weekly. The administering of the composition can include a drug holiday of one or more consecutive days during which the composition is not administered. The composition can be administered on a dosing schedule that includes administering: (a) daily for a period of 6 days followed by one day of no treatment, and repeating for 2 to 52 cycles of the schedule; or (b) daily for a period of 5 days followed by two days of no treatment, and repeating for 2 to 52 cycles of the schedule; or (c) daily for 4 days followed by one day of no treatment, and repeating for 2 to 73 cycles of the schedule; or (d) daily for 3 days followed by one day of no treatment, and repeating for 2 to 91 cycles of the schedule; or (e) daily for 2 days followed by one day of no treatment, and repeating for 2 to 122 cycles of the schedule; or (f) every other day for a period of 2 to 52 weeks. The composition can be administered for a period of 2 weeks to 12 months, or longer. In the compositions provided herein, a daily dosage of fullerene in the composition can be in the range of 20 mg to 80 mg. The administering can include providing a boost dose that includes a dosage of fullerene that is 10% to 400% higher than the daily dosage of fullerene. When administered, the boost dose can be administered at the beginning of each dosing schedule, or the end of each dosing schedule, or after 2, 3, 4, or more cycles of the dosing schedule.

Also provided is a method for producing a drug delivery vehicle, comprising complexing a therapeutic agent to the fullerene of the aqueous composition provided herein.

### DETAILED DESCRIPTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

### A. DEFINITIONS

Unless defined otherwise, all technical and scientific terms used herein have the same meaning as is commonly understood by one of skill in the art to which the inventions belong. All patents, patent applications, published applications and publications, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

As used herein, the singular forms “a”, “an” and “the” are intended to include the plural forms as well, unless the context clearly indicates otherwise.

As used herein, all ranges include the upper and lower limits. As used herein, the recitation of a numerical range for a variable is intended to convey that the variable can be equal to any value(s) within that range, as well as any and all sub-ranges encompassed by the broader range. Thus, the variable can be equal to any value or values within the numerical range, including the end-points of the range. As an example, a variable which is described as having values between 0 and 10, can be 0, 3, 4-8, 2.15, 6.8-9.1, etc.

As used herein, “about” is a term of approximation and is intended to include minor variations in the literally stated amounts, as would be understood by those skilled in the art. Such variations include, for example, standard deviations associated with techniques commonly used to measure the amounts of the constituent elements or components of an alloy or composite material, or other properties and characteristics. All of the values characterized by the above-described modifier “about,” are also intended to include the exact numerical values associated therewith. Hence “about 5 percent” means “about 5 percent” and also “5 percent.”

As used herein, “optional” or “optionally” means that the subsequently described event or circumstance does or does not occur, and that the description includes instances where the event or circumstance occurs and instances where it does not. For example, an optional component in a system means that the component may be present or may not be present in the system.

As used herein, the terms “comprises” and “comprising” are inclusive and open ended, and not exclusive. When used in the specification and claims, the terms “comprises” and “comprising” and variations thereof mean the specified features, steps or components are included, but do not exclude other features, steps or components.

Any compositions described herein are intended to encompass compositions which consist of, consist essentially of, as well as comprise, the various constituents identified herein, unless explicitly indicated to the contrary.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

As used herein, the term “exemplary” means “serving as an example or illustration,” and should not be construed as being preferred or advantageous over other configurations or formulation disclosed herein.

As used herein, the term “structured water” refers to a three-dimensional helical cage structure of polygonal water molecules having a hollow lumen, wherein the polygonal water molecules comprise two or more adjacent water molecules connected by hydrogen bridges. When viewed from the top, the arrangements of the water molecules of the helical cage structure has a hexagonal shape. The terms “structured water” and “H3O2 molecule” are used interchangeably through this application. The structure and growth of planar structures of water at different interfaces have been studied earlier. These previous studies are related to natural hydrogen bridge interactions in a particular zone of water, while the structured water of this invention is such that the arrangement of water molecules is altered by applying high energy processes to the water during the processes of cavitation and implosion in addition to the effects of magnetization and mineral injection processes. These processes change the energy of the bonds between adjacent water molecules, and a three-dimensional helical cage structure of polygonal water molecules having a hollow lumen, wherein the polygonal water molecules comprise two or more adjacent water molecules connected by hydrogen bridges with unique properties is achieved. The main differences between the “structured water” or H3O2 molecule found in the literature and that of this invention lies in the promotion of molecular self-replication, where the formation of the three-dimensional spiral cage structure of this invention, which is achieved under appropriate high energy processes, is promoted.

Moreover, the structured water of this invention is different from generally-known or described “structured water,” because the “structured water” used in the art refers to an intrinsic process of water. In comparison, the structured water of this invention is created by the application of high energy processes (“structuration”) as described herein. Structuration is a process in which, by means of implosion and cavitation energy, together with some organic and inorganic salts, at a temperature below atmospheric temperature, water is subjected to drastic changes of pressure and temperature in microstates so that this energy is able to enhance molecular interactions and change the properties of the water. As a result, the electrical and thermal conductivity of water can be changed to promote the formation of structured water of this invention. This change in the properties of water, together with the subsequent lowering of temperature, addition of molecular gases, and magnetization, promote the formation of the structured water of this invention. The structured water used in this invention changes the properties of the water and the bioavailability of its constituent elements. As used herein below, unless otherwise indicated, the term structured water refers to the structured water created by the application of high energy processes (“structuration”) as described herein.

As used herein, a “composition” and “formulation” are used interchangeably, and refer to an aqueous formulation suitable for administration to and consumption by a subject.

Unless indicated otherwise, each of the individual features or embodiments of the present specification are combinable with any other individual feature or embodiment that are described herein, without limitation. Such combinations are specifically contemplated as being within the scope of the present invention, regardless of whether they are explicitly described as a combination herein.

As used herein, “weight percent” or “wt %” refers to the concentration of a substance as the weight of that substance divided by the total weight of the composition and multiplied by 100.

As used herein, “surfactant” refers to a compound that lowers the surface tension between two liquids or between a liquid and a solid. Surfactants are typically amphiphilic, meaning they comprise both a hydrophilic moiety and a hydrophobic moiety, such as fatty alcohol groups and compounds that form micelles in an aqueous solution. Surfactants generally are classified depending on the charge of the surface active moiety, and can be categorized as cationic, anionic, nonionic and amphoteric surfactants.

As used herein, a “micelle-forming agent” is a compound that can be strongly absorbed onto a two-phase interface due to the hydrophilic-lipophilic balance of the compound, or that augments the ability of another compound to be absorbed onto a two-phase interface. Examples of micelle-forming agents include a surfactant, a liposoluble agent, an oil, and a combination thereof.

As used herein, “micelle” refers to a cluster of amphipathic molecules, such as a surfactant, formed when the molecules are dissolved in water at a certain concentration or more, in which the hydrophilic groups face the outside of the cluster while the lipophilic groups face the inside of the cluster.

As used herein, “room temperature” means an ambient temperature in the range of from about 20° C. to about 25° C. (generally having an average of about 21° C.).

As used herein, the terms “subject” and “patient” are used interchangeably herein, and refer to an animal, for example a human, to whom treatment with the composition according to the present invention, is provided. The term “subject” as used herein refers to human and non-human animals. The term “non-human animals” includes all vertebrates, e.g., mammals, such as non-human primates, (particularly higher primates), sheep, dogs, rodents (e.g. mice or rats), guinea pigs, goats, pigs, cats, rabbits, cows, and non-mammals such as chickens, amphibians, and reptiles. In one embodiment, the subject is human. In another embodiment, the subject is an experimental animal or animal substitute as a disease model. Examples of subjects include humans, dogs, cats, cows, goats, and mice.

### B. DESCRIPTION OF THE DRAWINGS

FIG. 1 is a plan view of one of the fullerene faces with the planar H3O2 structure.

FIG. 2 is an isometric view of a fullerene (center) surrounded by the planar structure of H3O2 molecules.

FIG. 3 is a schematic illustration of the structured water used in this invention, showing a two-dimensional ordered hexagonal matrix arrangement of the water molecules after the structuration process.

FIG. 4 is an illustration of the hexagonal arrangement of water molecules showing two contiguous planes of hexagonal formations of hydrogen and oxygen molecules where the plane of the water molecule is parallel to the surface.

FIG. 5A is an illustration of a single three-dimensional helical cage structure of polygonal water molecules of the structured water used in this invention, and FIG. 5B is a top view of the helical structure of FIG. 5A.

FIG. 6 is a visual representation of the arrangement of various cations within the hollow lumen of the structured water of this invention.

FIGS. 7A to 7C are illustrations representing the three phases during the structuration process of producing structured water that can be used in this invention.

FIGS. 8 and 9 are representative illustrations to explain the processes of cavitation and implosion.

FIG. 10 is a schematic illustration of the generation of H₂ from the reaction of Mg and H₂O.

FIG. 11 is a schematic illustration of obtaining metallic magnesium from naturally occurring magnesite using electrolysis and precipitation, and the generation of H₂ from the reaction of Mg and H₂O.

FIGS. 12-17 are illustrative water structuring systems that can be used to produce structured water that can be used in the compositions of this invention.

FIG. 18A is an illustration of an exemplary water structuring system, and FIG. 18B is an exploded view of the water structuring system of FIG. 18A. FIGS. 18C-18E are illustrations of various components of the water structuring system of FIG. 18A.

FIGS. 18F and 18G are representative illustrations of a vortex generated inside the water structuring system of FIG. 18A.

FIGS. 19A and 19B are illustrations of a large-scale water structuring system that can be used to produce structured water that can be used in the compositions of this invention.

FIGS. 20A-20C are illustrations of a compact water structuring system that can be used to produce structured water that can be used in the compositions of this invention.

FIG. 21 is a cutaway view of section 2000A of the water structuring system of FIG. 18A.

FIG. 22 is a flowchart of a method for forming structured water that can be used in the compositions of this invention.

FIG. 23 is a schematic representation of a device for producing fullerenes and micelles.

FIG. 24 is a schematic representation of a device for producing an aqueous composition containing fullerene, hydrogen gas and minerals as described herein.

### C. DESCRIPTION OF THE COMPOSITIONS PROVIDED HEREIN

The aqueous compositions provided herein include fullerene in combination with structured water containing dissolved hydrogen gas, minerals and additives. The fullerene is solubilized in the water by forming micelles that surround the fullerene. The micelles are structures in which surfactant molecules are organized in a surface layer surrounding hydrophobic fullerene molecules to solubilize them in water. The micelles form a spherical structure in which the hydrophilic surface layer is in contact with water and the hydrophobic core layer encapsulates the fullerene molecules. Thus, the solubilization of fullerene in water through the formation of micelles allows the dispersion of the hydrophobic fullerene molecule in an aqueous solution, which can facilitate its use in biological and medical applications.

The structured water, containing hydrogen and minerals, can be produced by the process described in U.S. patent application Ser. No. 18/100,562, filed by BEST PLANET SCIENCE LLC on Jan. 23, 2023, and in U.S. patent application Ser. No. 18/100,563, filed by BEST PLANET SCIENCE LLC on Jan. 23, 2023, the entire disclosure of each of which is incorporated herein by reference.

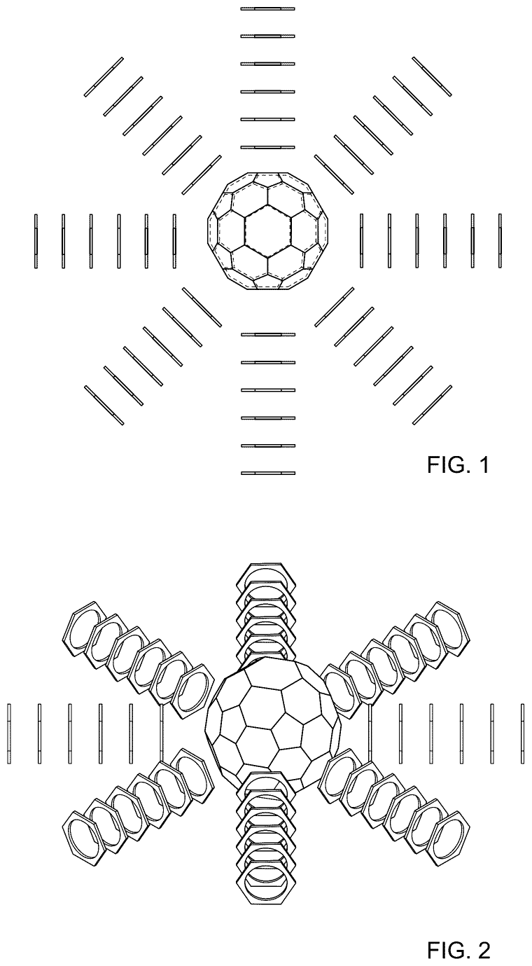

Fullerene is a vehicle that improves properties such as bioavailability of the different minerals and hydrogen contained within the structured water, and also improves electrical and thermal conductivity properties which promotes cellular interaction with these molecules. The fullerene can improve interaction of minerals and hydrogen with different types of cells in the body. The structure of the fullerene dispersed in the structured water, as shown in FIG. 1, avoids the drawbacks of the conventional procedures used in the art to solubilize and stabilize fullerene in aqueous solutions. In particular, the use of charge transfer complexes is avoided, and bioavailability is improved. When present, the effect of organic solvents on the fullerene is improved due to the structure of water.

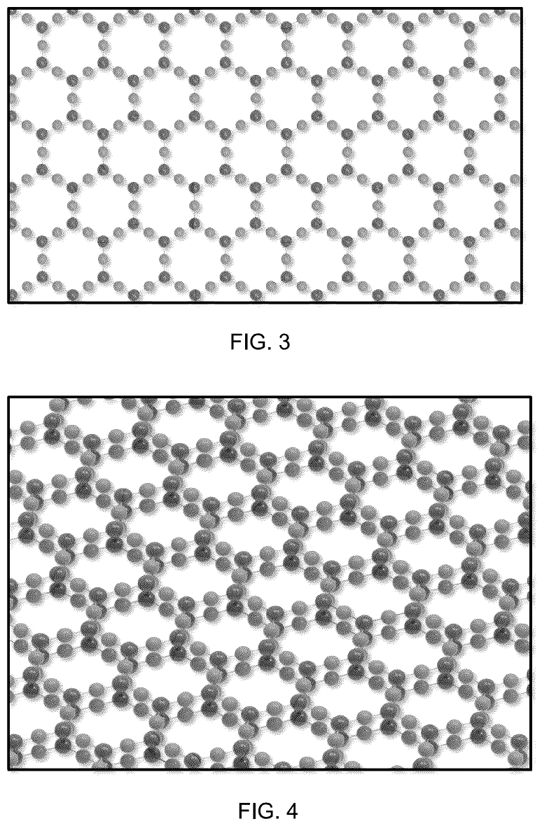

FIG. 1 shows a schematic representation of a top view of the fullerene macromolecule surrounded by the structured water with minerals and hydrogen, showing the H3O2 structure. The surfactant is omitted from the figure for simplification. As can be seen in FIG. 1, a fullerene macromolecule in the center is surrounded by H3O2 hexagonal structures of the structured water (the hexagons that form the planar molecule H3O2). FIG. 2 shows schematically an isometric view of the fullerene surrounded by the hexagons that form the structure of H3O2. The use of fullerene in structured water promotes the formation of planar H3O2 molecules, which allow a better arrangement of the organic material. FIG. 1 and FIG. 2 show novel H3O2 arms surrounding a C60 fullerene having hexagonal moieties.

Because the aqueous composition provided herein containing structured water results in the constituents having a better bioavailability, the aqueous composition can be designed to be administered orally or nonparenterally. As administered, the compositions include a micelle-coated fullerene stabilized due to the structured water, dilute hydrogen as a gas, and minerals. In the composition, the fullerene encapsulated with micelles is dissolved in the aqueous substance because of the micelles. In some formulations, the fullerene is encapsulated with a surfactant, which forms the micelles.

### Fullerene

Fullerene compounds are a novel class of molecules under development for various biomedical applications (Hardt et al., European Journal of Drug Metabolism and Pharmacokinetics 43(5): 543-554 (2018)), especially against cancer. Fullerenes include non-modified fullerenes and modified fullerenes. Research has identified multiple mechanisms by which fullerene nanoparticles exert their antitumor activity. For example, [C(COOH)₂]₃ carboxyfullerene has been shown to possess antioxidant and prooxidant activity, which may contribute to its effectiveness in cancer therapy (Rajagopalan et al., Antimicrobial Agents and Chemotherapy 40(10): 2262-2265 (1996)). In addition, interaction studies with cancer cells have shown a significant reduction in the IC50 value in cells treated with fullerene nanoparticles, indicating a higher cytotoxic efficacy compared to other forms of treatment.

Fullerene nanoparticles have shown promising antitumor activity, which is attributed to several mechanisms. For example, fullerene nanoparticles can generate reactive oxygen species (ROS), such as free radicals, which have cytotoxic effects on cancer cells. This process can induce apoptosis and suppress tumor cell proliferation. This phenomenon is due to the ability of fullerenes to interact with molecular oxygen and form ROS, such as superoxide and hydrogen peroxide, which can damage cancer cells and cause their death.

Reactive oxygen species (ROS) are produced as a consequence of normal physiological aerobic metabolism. The mitochondrial electron transport chain, peroxisomes, NADPH oxidase, uncoupled nitric oxide synthetase, and the cytochrome P450 system are the most important sources of ROS production. The imbalance between the production of ROS and the antioxidant defense system in living systems causes a breakdown in cellular function and damage. This imbalance occurs due to an overproduction of ROS and a reduction in the antioxidant defense mechanism. Protective actions against ROS are carried out by several enzymes (superoxide dismutase, catalase, and glutathione peroxidase) and also by non-enzymatic compounds (vitamin E, ascorbate, glutathione, transferrin, ceruloplasmin, etc.). ROS are crucial modulators of cellular functions. At low concentrations, ROS are essential participants in cell signaling, induction of the mitogenic response, defense against infectious agents, while excess ROS can alter normal cellular function and promote irreversible damage to lipids, nucleic acids and cellular proteins. ROS, especially H₂O₂, serve as messenger molecules through oxidative modification of signaling proteins. Thus, a balance between the production of ROS and their removal allows normal cellular function, while an imbalance causes oxidative stress with pathological consequences.

In 1931, scientist Otto Heinrich Warburg (1883-1970) received the Nobel Prize for discovering the primary cause of cancer in his thesis “The primary cause and prevention of cancer.” According to Mr. Warburg, cancer is the consequence of an anti-physiological diet and an anti-physiological lifestyle. An anti-physiological diet (diet based on acidifying foods and a sedentary lifestyle) creates an acidic environment in our body and this, in turn, causes the expulsion of oxygen from the cells. Mr. Warburg stated:
“Lack of oxygen and acidosis are two sides of the same coin: when you have one, you have the other.”
“Acidic substances reject oxygen; On the other hand, alkaline substances attract oxygen.”
“Depriving a cell of oxygen for 48 hours can make it cancerous.”
“All normal cells have an absolute requirement for oxygen, but cancer cells can live without oxygen (this is a rule without exception).”
“Cancerous tissues are acidic tissues, while healthy tissues are alkaline tissues”

In his work “The Metabolism of Tumors in the Body” (Journal of General Physiology 8(6): 519-530 (1927)) Warburg demonstrated that all forms of cancer are characterized by two basic conditions: acidosis and hypoxia (lack of oxygen). He also discovered that cancer cells are anaerobic and cannot survive in the presence of high levels of oxygen. Instead, they survive thanks to glucose as long as the environment is free of oxygen.

Therefore, cancer would be nothing more than a defense mechanism that certain cells in the body have to continue living in an acidic environment lacking oxygen. Healthy cells live in an alkaline and oxygenated environment, which allows them to function normally. Once the digestion process is complete, the food will generate a condition of acidity or alkalinity in the body depending on the quality of the proteins, carbohydrates, fats, minerals and vitamins consumed. The acidifying or alkalizing result is measured through pH. It is important to know how acidic foods and alkaline foods affect health, since for cells to function correctly and adequately their pH must be slightly alkaline. In a healthy person, the pH of the blood is between 7.40 and 7.45. Keep in mind that if the blood pH fell below 7 we would enter a coma close to death.

It has been observed that fullerene nanoparticles can interfere with tumor cell proliferation by affecting different cell signaling pathways. This can stop the growth of tumors and reduce their size. Fullerene nanoparticles can activate intracellular signaling pathways that lead to apoptosis, or programmed cell death, in cancer cells. This process selectively eliminates tumor cells without harming surrounding healthy cells. It has been suggested that fullerene nanoparticles can modulate the tumor microenvironment by inhibiting angiogenesis, the formation of new blood vessels necessary for tumor growth, and reducing tumor invasion and metastasis. Fullerene nanoparticles can interact with various biomolecules and cellular components, such as proteins, lipids and nucleic acids, which can alter cellular function and affect the viability of tumor cells. These mechanisms suggest that fullerene nanoparticles have great potential as therapeutic agents in cancer treatment. However, difficulty in delivery of the fullerene nanoparticles as an active agent has hampered exploiting these mechanisms. The compositions provided herein address this deficiency and allow the fullerene nanoparticles to be effectively delivered by non-parenteral administration, particularly via oral administration. The compositions provided herein also have been observed to improve the antitumor efficacy of fullerene nanoparticles when administered to patients having cancer.

Fullerene nanoparticles can be internalized by cancer cells through several mechanisms, such as receptor-mediated endocytosis, pinocytosis, and phagocytosis. Once internalized, fullerene nanoparticles can localize in different cellular compartments, such as the cytoplasm, nucleus and mitochondria. This intracellular localization can influence the therapeutic effects of the nanoparticles and their ability to induce apoptosis or stop cell proliferation. The internalized fullerene nanoparticles can interact with cellular organelles and biomolecules.

Endocytosis is a general term for the different types of active transport that introduce particles into a cell by enclosing them in plasma membrane vesicles. Receptor-mediated endocytosis is a form of endocytosis in which receptor proteins on the surface of the cell are used to capture a certain target molecule. When receptors bind to their target molecule, endocytosis is triggered, and the receptors, along with their attached molecules, are absorbed into the cell in a vesicle. Coating proteins participate in this process by giving the vesicle its rounded shape and helping it detach from the membrane. Receptor-mediated endocytosis allows cells to take up large quantities of molecules that are relatively scarce (present in low concentrations) in the extracellular fluid.

Phagocytosis is a type of endocytosis and is the process by which a cell uses its plasma membrane to engulf a large particle (≥0.5 μm), giving rise to an internal compartment called phagosome. Phagocytosis allows for the introduction of large particles, such as cells or cell debris, into the cell stored in a large vacuole. Pinocytosis is a form of endocytosis in which a cell absorbs small amounts of extracellular fluid. Pinocytosis occurs in many types of cells and occurs continuously as the cell samples the surrounding fluid over and over again to obtain all the nutrients and other molecules present. The pinocytosed material is stored in small vesicles, much smaller than the large vacuole produced by phagocytosis.

Once internalized, fullerene nanoparticles can localize in different cellular compartments, such as the cytoplasm, nucleus, and mitochondria. This intracellular localization can influence the therapeutic effects of the nanoparticles and their ability to induce apoptosis or stop cell proliferation. Fullerene nanoparticles can interact with different proteins and signaling molecules within cancer cells, which can modulate various cell signaling pathways associated with proliferation, survival, and apoptosis. This modulation can have significant effects on cancer cell viability and behavior. It has been shown that fullerene nanoparticles can induce apoptosis in cancer cells by activating intracellular signaling pathways associated with programmed cell death. In addition, they can also arrest the cell cycle at different stages, which limits the proliferation of tumor cells. Fullerene nanoparticles can influence gene expression in cancer cells by modulating transcription and translation of specific genes related to cell proliferation, apoptosis, and angiogenesis. Fullerene nanoparticles have demonstrated potent antitumor activity in preclinical studies, inducing apoptosis in cancer cells, inhibiting cell proliferation and reducing tumor growth in animal models. It has been suggested that fullerene nanoparticles can modulate the tumor microenvironment by inhibiting angiogenesis and reducing tumor invasion and metastasis, which could improve the efficacy of other cancer treatments. Taken together, the interaction between fullerene nanoparticles and cancer cells is a process that can influence multiple aspects of cell behavior and tumor viability. Despite their ability to induce cytotoxic effects in cancer cells, fullerene nanoparticles have shown low toxicity in normal cells and healthy tissues, making them promising candidates for the development of treatments with minimal side effects. Fullerene nanoparticles can be engineered to have enhanced tumor specificity by conjugation with ligands specific for receptors overexpressed on cancer cells, allowing selective delivery of therapeutic agents to tumors while minimizing effects on healthy tissues. Fullerene nanoparticles can serve as efficient carriers for the delivery of drugs and therapeutic agents to tumors, enhancing their solubility, stability and bioavailability and allowing a controlled and targeted release at the tumor site.

Fullerene stability has both thermodynamic and kinetic aspects. Kinetic stability is a concept that is linked to the average time that a molecule maintains its structure and function under certain conditions. When a carbon allotrope such as fullerene remains functional for a long period of time that allows it to carry out its function, it is said to be kinetically stable. On the other hand, thermodynamic stability refers to the conditions at which a process meets the thermodynamic criterion of spontaneity. The observed structural characteristics of many weak (non-covalent) interactions are characterized by a change in the Gibbs free energy when passing from the initial to the final state. This energy difference between the initial state and other intermediate or final conformations allows us to analyze and understand the thermodynamic stability of the fullerene.

Fullerenes exhibit antioxidant activity and free radical scavenging. Free radicals are unstable molecules that are a product of normal cell metabolism, however they can suffer from accumulation or other problems that are associated with cancer and other diseases. C60 fullerene has been shown to be a scavenger of these radicals, which consequently improves the environment of the cell and even within the cell. In addition, its effect can be enhanced with functional groups, which improve properties such as solubility. Compared to other antioxidants, fullerene has been shown to be more effective than vitamin E in inhibiting lipid peroxidation. The most prevalent fullerene, C60, is a stable icosahedron with 30 double bonds that readily accept free radicals, hence, it has been given the term “free radical sponge” (Kratschmer et al., Nature 347: 354-358 (1990)). This unique feature of C60 fullerene allows it to absorb cell-damaging reactive oxygen species, as well as its antioxidant action coupled with its small size and large surface area, is what makes it attractive for efficient application in biomedicine and clinical therapy (Markovic et al., Biomaterials 29: 3561-3573 (2008)). Normal cellular processes and some abnormal reactions in mitochondria can lead to the generation of free radicals. Excess production of these free radicals can cause cellular damage and eventually lead to cancer. The intrinsic free radical scavenging property of fullerene is a breakthrough in cancer treatment. Due to the small size, fullerenes show higher permeability and retention effect (RPE) in the tumor mass, decreasing the concentration of reactive oxygen species (ROS), without being consumed. It also inhibits the activation of cancer and tumor growth promoting genes and the formation of blood vessels (angiogenesis) that nourish tumor masses as an anticancer action (Chen et al., Theranostics 2: 238 (2012), DOI: 10.7150/THNO.3509). One application of the aqueous solutions provided herein is as a therapeutic that exhibits antioxidant activity and free radical scavenging.

Fullerene can also be oxidative under some circumstances, such as in use in photodynamic and photothermal therapies. Therefore, fullerenes can be applied as possible photosensitizers for photodynamic therapy (PDT). PDT is a noninvasive treatment that involves the generation of reactive oxygen species in a localized area, which is irradiated with electromagnetic radiation of a select wavelength or range of wavelengths, leading to the destruction of target cells, such as cancer cells, through various pathways. One application of the aqueous solutions provided herein is as a therapeutic for photodynamic therapy.

In recent decades, nanoparticles have gained prominence in the treatment of cancer in combination with conventional therapies such as chemotherapy, surgery, radiotherapy, immunotherapy, and hormone therapy. In the conventional treatment of cancer, despite the many options currently available, the deleterious side effects that are common in their use, including alopecia, continue to be seen as deleterious. Another important phenomenon in the use of existing chemotherapeutics in cancer corresponds to the resistance to them and in some cases their low effectiveness. This can result in the need to change treatment regimes several times. Many existing chemotherapeutic regimes use therapeutic substances that are not specific for tumor cells, but can compromise the life and division of healthy cells with which the compounds come into contact. This situation not only makes it necessary to search for therapeutic options that continue to be effective in the control of neoplastic processes, but also to significantly reduce these side effects in their use. Other mechanisms in cancer therapy using fullerenes is that fullerenes can act as inhibitors of metastasis, decreasing the possibility of spread or cancer cells, free radical scavenging that results in protection of cell membranes of healthy cells. It has been shown in studies that fullerene derivatives exhibit a wide range of antitumor properties, including immune boosting, antioxidation, anti-metastasis, cell cycle arrest, suppression of tumor angiogenesis, and inhibition of multidrug resistance, making it an emerging option in the effective treatment of various types of cancer with fewer side effects and adverse reactions in its application. One application of the aqueous solutions provided herein is as a therapeutic in the treatment of cancer.

Another application of the aqueous compositions provided herein is as a drug delivery vehicle. Drug delivery with fullerenes can improve drug pharmacokinetics due to this nanomaterial that has the property of being bioactive and has the possibility of reaching the cell nucleus, where this nanoparticle can be used for selective drug delivery and controlled release due to its biocompatibility. This is shown in applications related to the quenching of reactive oxygen species and targeted imaging with functionalized fullerenes. Fullerene nanoparticles can serve as efficient carriers for the delivery of drugs and therapeutic agents to tumors, enhancing their solubility, stability, and bioavailability, and allowing a controlled and targeted release at the tumor site.

Another application of the compositions provided herein is as a biosensor. Fullerenes can be used for serum protein profiling as a material-enhanced laser desorption/ionization (MELDI) material for biomarker discovery. The recognition site responds to the presence of biomolecules and the transducer converts it into signals that can be measured. Fullerenes can be used as signal amplifiers, considering their electromagnetic fields. In some applications, an efficient mediator must be hydrophilic, therefore it must possess functional groups, which help it to conjugate biomolecules. Carboxylic acid, amine and hydroxyl groups stand out as functional groups for this application. The fullerene can be a polyhydroxylated fullerene, also known as fullerenol. Derivatized fullerene can cross the cell membrane. As an electron mediator, fullerene, modified or unmodified in the aqueous solutions provided herein, can be used in biosensors.

Fullerenes also have been the subject of research in the healthcare field regarding their use for diagnostic imaging and treatment. One approach was focused on the use of fullerene in magnetic resonance imaging, which use a gadolinium-based contrast medium, which can be encapsulated, because the fullerenes can trap metal atoms, and after injection the C60 fullerene can be distributed in the tissues and can relocate specifically in the targeted organs, without producing signs of toxicity. Because the formulations provided herein are aqueous, the fullerene is easily absorbed by the reticuloendothelial system. One application of the aqueous compositions provided herein is as a diagnostic imaging composition.

Any fullerene known in the art can be used in the aqueous formulations provided herein. There are many types of fullerenes, characterized by always having 12 pentagons, regardless of the number of hexagons. The smallest fullerene is C20, which contains 20 pentagons and no hexagons. However, this type of structure has strong internal stresses because the shape of each carbon molecule is strongly nonplanar. More stable fullerenes are C28, C32, C44, C44, C50, C58, C60, C70, C76, C84, C240, C540, C960 and many others. Of the forms of fullerenes, the most commonly used for exploration in medical applications are C60 and C70, and have been shown to exhibit therapeutic efficacies and have easy renal elimination.

Fullerenes are commercially available. Production of fullerenes via electric arc and combustion are the most developed technologies, and fullerenes are produced at kg scale worldwide. Electric arc manufacturing has become an attractive technology because the cost of the reactor is low compared to combustion, and it is a green process despite requiring higher energy consumption. In addition, electric arc does not produce carcinogenic by-products such as polycondensed aromatic hydrocarbons that are produced by the combustion method, which requires additional intensive purification stages.

The fullerene selected can be unmodified, or can be modified. A modified fullerene can include one or more hydrophilic groups on a surface of the molecule. Any group known in the art to impart hydrophilicity can be used to surface-modify the fullerene molecule. Exemplary groups include, but are not limited to, hydroxyl groups, carboxylic acids, alcohols, amines, or sulfonates. The surface-modifying groups can be attached to the surface of the fullerene via covalent or non-covalent bonds. Surface modification with hydrophilic groups can allow the fullerenes to dissolve in water and disperse in solution. The fullerene can be a polyhydroxylated fullerene, known as a fullerenol.

A modified fullerene can be formed by complexing the fullerene with one or a combination of water-soluble polymers. Examples of such polymers include, but are not limited to, a polyethylene glycol (PEG), a dextran, a polyvinyl alcohol, a polysaccharides, and a protein, and are attached to the fullerene surface by non-covalent bonds. Complexes formed with water-soluble polymers are stable in solution and allow the fullerenes to dissolve and disperse in water.

The amount of the fullerene in the aqueous composition can be an amount from about 1 mg/L to 30,000 mg/L. The aqueous composition can be diluted to deliver a desired dose. The aqueous composition provided herein can include an amount of fullerene, which can be modified or unmodified or a combination of both, to deliver a dose containing 10 mg to 100 mg fullerene. The aqueous composition provided herein can include an amount of fullerene, which can be modified or unmodified or a combination of both, to deliver a dose containing 10 mg fullerene, 15 mg fullerene, 20 mg fullerene, 25 mg fullerene, 30 mg fullerene, 35 mg fullerene, 40 mg fullerene, 45 mg fullerene, 50 mg fullerene, 55 mg fullerene, 60 mg fullerene, 65 mg fullerene, 70 mg fullerene, 75 mg fullerene, 80 mg fullerene, 85 mg fullerene, 90 mg fullerene, 95 mg fullerene, or 100 mg fullerene.

### Structured Water

The structured water used herein, containing hydrogen and minerals, can be produced by the process described in U.S. patent application Ser. No. 18/100,562, filed by BEST PLANET SCIENCE LLC on Jan. 23, 2023, and in U.S. patent application Ser. No. 18/100,563, filed by BEST PLANET SCIENCE LLC on Jan. 23, 2023, the entire disclosure of each of which is incorporated herein by reference. The structured water also can be produced using other methods described below.

Cavitation bubbles can appear within a fluid when a vortex is generated in a fluid by the action of a rotor (e.g., rotating blade). As these cavitation bubbles encounter the pressure differential created by the vortex along isobaric lines, the cavitation bubbles implode into an elliptical-shaped imploded cavitation bubble. As described below, a vortex-generating device can be used. The vortex can generate an environment of microstates, which facilitate cavitation and implosion processes resulting in a localized pressure, calculated to be about 0.2 GPa to about 3 GPa and a localized temperature, calculated to be at least 5000 K in the water that facilitates the formation of structured water. As one example, the vortex can be created by rotating a vortex-generating system at 3600 rpm, which generates an average linear speed of about 50 m/s of the water in the vortex, and an absolute pressure that is less than 2 kPa.

### Surfactants

Because the solubility of fullerene in water is low (10⁻⁶ mg/L) and the base product is water which contains organic and inorganic salts, integration of the fullerenes was performed by creation of micelles that surround the fullerene. The micelles can be formed by the use of a surfactant, a liposoluble agent, an oil, or a combination thereof. In some formulations, the micelles can be formed by the use of a surfactant alone. In some formulations, the micelles can be formed by the use of an oil alone. In some formulations, the micelles can be formed by the use of a surfactant in combination with an oil. Exemplary oils include an essential oil, such as a flavor, an olive oil, and a coconut oil. In some formulations, the micelle was formed with an essential oil, which can be a low density material, resulting in a nanoemulsion that at the same time can impart a pleasant flavor to the product. Exemplary essential oils include, but are not limited to, thymol, menthol, methyl salicylate (wintergreen oil), eucalyptol, carvacrol, camphor, anethole, carvone, eugenol, isoeugenol, limonene, n-decyl alcohol, citronel, α-salpineol, methyl acetate, citronellyl acetate, isoamyl acetate, methyl eugenol, cineol, linalool, ethyl linalool, spearmint oil, peppermint oil, lemon oil, orange oil, sage oil, rosemary oil, cinnamon oil, pimento oil, laurel oil, cedar leaf oil, geraniol, verbenone, anise oil, bay oil, benzaldehyde, bergamot oil, bitter almond, chlorothymol, cinnamic aldehyde, citronella oil, clove oil, eucalyptus oil, guaiacol, lavender oil, pine oil, pine needle oil, sassafras oil, spike lavender oil, thyme oil, clove oil and combinations thereof

Surfactants are amphiphilic molecules, meaning that they have a hydrophobic and a hydrophilic part. When surfactants are added to a solution containing fullerenes, the hydrophilic part of the surfactant interacts with the water molecules, while the hydrophobic part of the surfactant interacts with the fullerenes. This can lead to the formation of micelles or surfactant-fullerene complexes that are soluble in water.

Any surfactant known in the art can be used. In some applications, a nonionic surfactant is used to interact with the fullerene. The nonionic surfactant can be selected from among a sorbitan ester, a polyalkoxylated sorbitan fatty acid ester, a polyoxyethylene ether, a polysorbate, or a combination thereof. In some formulations, a polysorbate surfactant is used. Polysorbate surfactants are ethoxylated sorbitan esters and include polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), and polysorbate 80 (polyoxyethylene (20) sorbitan monooleate), wherein the number 20 following the “polyoxyethylene” part refers to the total number of oxyethylene-(CH₂CH₂O)— groups found in the molecule. The number following the “polysorbate” part is related to the type of fatty acid associated with the polyoxyethylene sorbitan part of the molecule. Monolaurate is indicated by 20, monopalmitate is indicated by 40, monostearate by 60, and monooleate by 80. In some formulation, the fullerenes are coated with the nonionic surfactant polysorbate 80.

In some formulations, an olive oil can be present in an amount of 0.1 to 800 g/L, or in an amount of 1 to 80 g/L. In some formulations, a coconut oil can be present in an amount of 0.1 to 800 g/L, or in an amount of 1 to 80 g/L. In some formulations, a surfactant can be present in an amount of 1 to 10,000 mg/L, or in an amount of 10 to 1,000 mg/L. In some formulations, the surfactant can be or include a polysorbate. In some formulations, an essential oil can be present in an amount of 0.01 to 20 g/L, or in an amount of 0.1 to 5 mg/L. In some formulations, the essential oil includes benzoic aldehyde (benzaldehyde).

In some methods, the aqueous composition containing fullerene can be prepared by a process that includes the preparation and solubilization of the fullerene, the preparation of the aqueous substance with minerals and hydrogen, and the mixing of these two components together under a structuring method described in U.S. patent application Ser. No. 18/100,562, filed by BEST PLANET SCIENCE LLC on Jan. 23, 2023, and in U.S. patent application Ser. No. 18/100,563, filed by BEST PLANET SCIENCE LLC on Jan. 23, 2023, the entire disclosure of each of which is incorporated herein by reference. Additional hydrogen gas can be added.

When water is structured, it increases the capacity to retain dissolved hydrogen and change its diamagnetic properties compared to traditional water. The maximum retention capacity of traditional drinking water for dissolved hydrogen is about 2 ppm. In comparison, structured water can retain dissolved hydrogen in amounts of about 3 ppm to about 5 ppm. That is, structured water increases retention capacity of hydrogen by about 50% to about 150% compared with traditional drinking water.

As used herein, the term “structured water” refers to a three-dimensional helical cage structure of polygonal water molecules having a hollow lumen, wherein the polygonal water molecules comprise two or more adjacent water molecules connected by hydrogen bridges. When viewed from the top, the arrangements of the water molecules of the helical cage structure has a hexagonal shape. The terms “structured water” and “H₃O₂ molecule” are used interchangeably through this application. As described earlier, the structure and growth of planar structures of water at different interfaces have been studied earlier. These previous studies are related to natural hydrogen bridge interactions in a particular zone of water, while the structured water of this invention is such that the arrangement of water molecules is altered by applying high energy processes to the water during the processes of cavitation and implosion in addition to the effects of magnetization and mineral injection processes, as described herein. These processes change the energy of the bonds between adjacent water molecules, and a three-dimensional helical cage structure of polygonal water molecules having a hollow lumen, wherein the polygonal water molecules comprise two or more adjacent water molecules connected by hydrogen bridges with unique properties is achieved. The main differences between the “structured water” or H₃O₂ molecule found in the literature and that of this invention lies in the promotion of molecular self-replication, where the formation of the three-dimensional spiral cage structure of this invention, which is achieved under appropriate high energy processes, is promoted.

Moreover, the structured water used in this invention is different from generally-known or described “structured water,” because the “structured water” previously known refers to an intrinsic process of water. In comparison, the structured water used in this invention is created by the application of high energy processes (“structuration”) as described herein. Structuration is a process in which, by means of implosion and cavitation energy, together with some organic and inorganic salts, at a temperature below atmospheric temperature, water is subjected to drastic changes of pressure and temperature in microstates so that this energy is able to enhance molecular interactions and change the properties of the water. As a result, the electrical and thermal conductivity of water can be changed to promote the formation of structured water of this invention. This change in the properties of water, together with the subsequent lowering of temperature, addition of molecular gases, and magnetization, promote the formation of the structured water of this invention. The structured water used in the compositions of this invention changes the properties of the water and the bioavailability of its constituent elements.

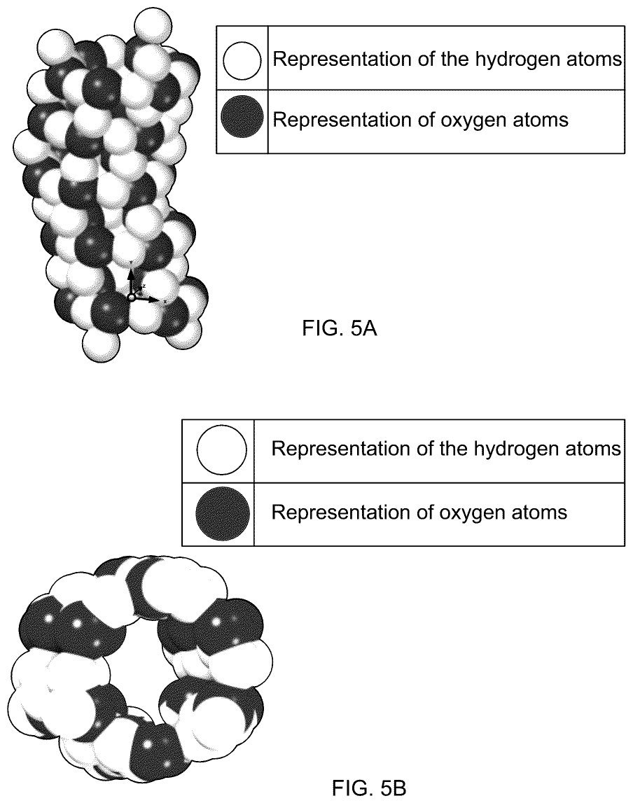

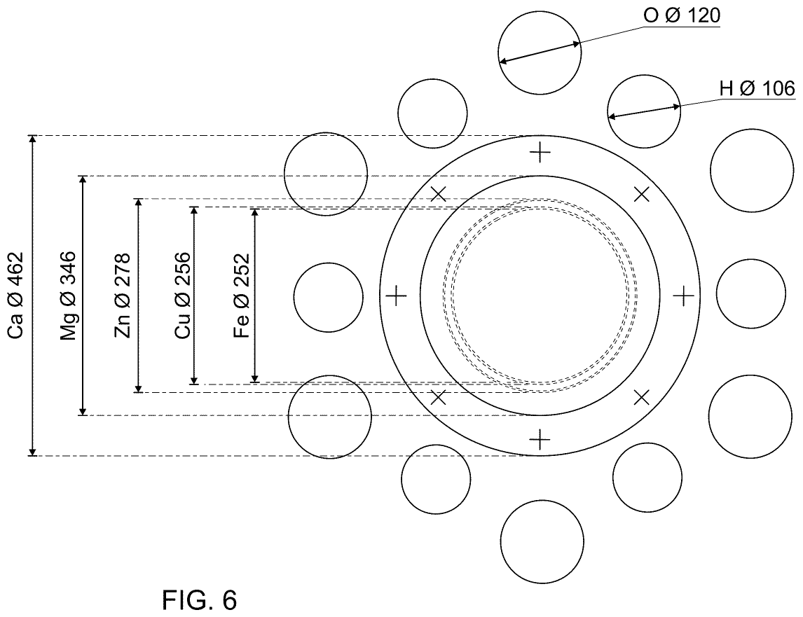

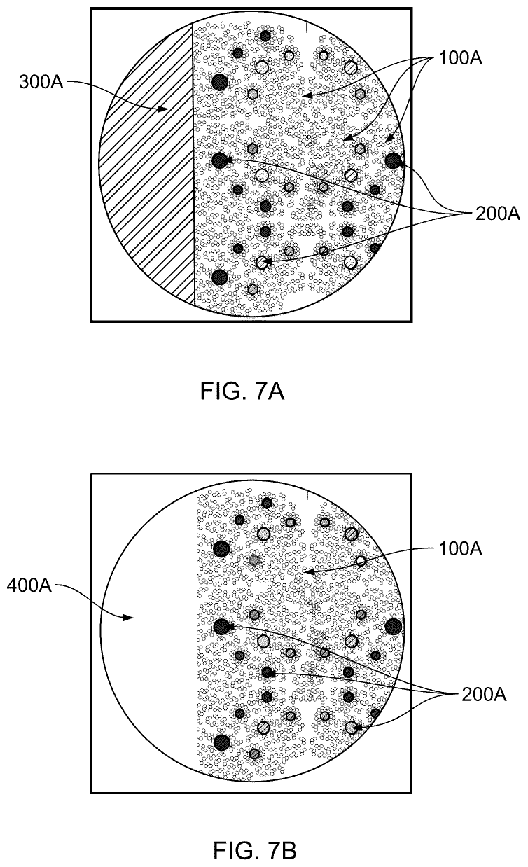

For example, in the structured water, adjacent molecules are joined by means of hydrogen bridges to form a hexagonal structure as shown in FIGS. 3, 4, 5A and 5B. FIG. 3 is a schematic illustration of a two-dimensional ordered hexagonal matrix arrangement of water molecules where the pattern is replicated in the different planes, and this formation is considered superior over the general arrangement of water molecules, and allows the density of the fluid to decrease in addition to the change of electromechanical properties. FIG. 4 is an illustration of the hexagonal arrangement of water molecules showing two contiguous planes of hexagonal formations of hydrogen and oxygen molecules where the plane of the water molecule is parallel, or substantially parallel, to the surface. FIG. 5A is an illustration of a single three-dimensional helical cage structure of polygonal water molecules, wherein the polygonal water molecules comprise two or more adjacent water molecules connected by hydrogen bridges. FIG. 5B is a top view of the single spiral cage structure of FIG. 5A showing the hexagonal shape of the three-dimensional helical cage structure. FIG. 5B shows a single three-dimensional helical cage structure of this invention and has the measurement of the atomic radii to scale, as estimated for the given thermodynamic conditions discussed herein. Although FIG. 5B is a top view of a single helical cage structure, multiple representations of the water molecules occur.

Multiple hexagonal structures formed by adjacent water molecules can be stacked in a direction perpendicular to the plane that forms the hexagonal structure. Each of the hexagonal structures forming the stacked structure can be rotated due to its electromagnetic properties. The arrangement of the hexagonal structures formed by the H₃O₂ molecule can also be replicated in different planes, which allows an increase in the density of the fluid in addition to the change of electromechanical properties. In other arrangements, two contiguous planes of hexagonal structures can be formed, as shown in FIG. 2. The structured water can include multiple water molecules in a planar orientation where adjacent water molecules are joined by hydrogen bridges forming hexagonal rings of water molecules forming a plane of a two-dimensionally ordered hexagonal matrix arrangement of water molecules, which is replicated in a plurality of planes stacked in a direction perpendicular to the plane of the of two-dimensionally ordered hexagonal matrix arrangement and connected via hydrogen bridges to form multiple layers of the two-dimensionally ordered hexagonal matrix arrangement, forming a plurality of three-dimensional helical cage structures of polygonal water molecules, wherein each of the helical cage structures has a central hollow lumen, and when viewed from a top, each of the helical cage structures has a hexagonal shape. A density of the structured water can be 10% higher than a density of standard water. A density of the structured water can be about 1.5 to about 5 times a density of standard water.

The stability and resultant properties of the structured water formed by the interaction of adjacent water molecules is a result of the electromagnetic effects between the molecular hydrogen and the H₃O₂ structure of this invention. This structure forms a matrix that has the ability to weave a network capable of trapping the hydrogen molecule within the hollow lumen formed in the three-dimensional cage structure of the H3O₂. This arrangement imparts buoyancy forces to the H₃O₂ structure and reduces or maintains, but does not increase, any forced entanglement between the adjacent water molecules. This behavior can be explained by the Zeeman/Stark effect, where, despite the small electromagnetic field exerted by the atoms on the water molecules, they affect the energy levels around them and change as described by these phenomena.

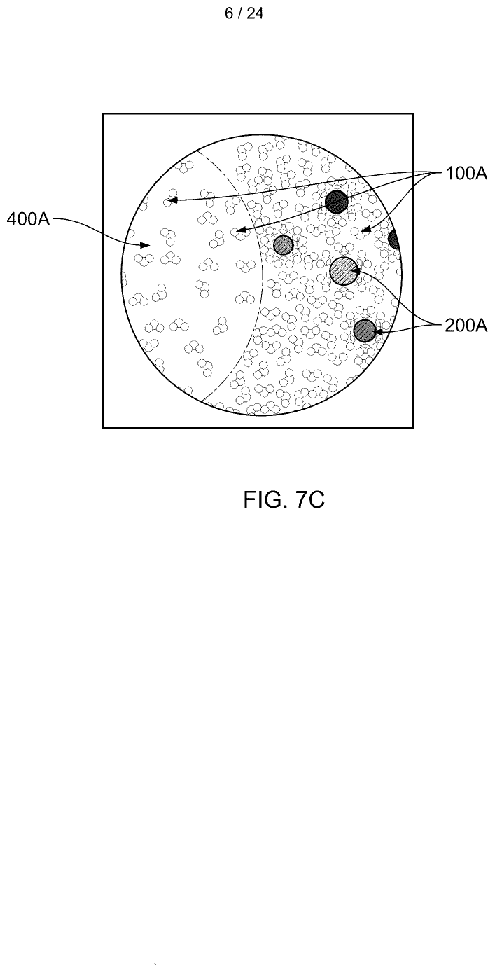

The hexagonal structures formed by hydrogen bridging between adjacent water molecules results in a stabilized material and different salts can adhere to the surface of the stabilized material. As illustrated in FIG. 6, the size and structure of various organic salts of the minerals is such that they can be accommodated within the three-dimensional helical cage structure of the H₃O₂ molecule of this invention. The structured water used in the compositions of this invention preferably comprises a material that includes metals (such as, but not limited to, calcium, magnesium, iron, zinc, copper, and selenium) and their salts.

The phenomenon of the formation of vapor in a fluid by a sudden decrease in pressure is known as cavitation. For this process the liquid is subjected to temperature above 5000° C. and pressures above 10 MPa. These temperature and pressure values are achieved from the potential energy of an implosion of water-vapor bubble and the kinetic energy of the fluid. The potential energy is established based on the specific pressure and volume parameters of each molecule, and is equal to the work generated by a pressure difference P_{d}−Pᵥ on its vapor volume throughout the collapse of the cavitation bubble, wherein Pa is the impeller (rotor) pressure and Pᵥ is the vapor pressure of the cavitation bubble. The implosion energy of an undisturbed vapor bubble is equal to the ambient pressure p^{∞}, as shown in Function 1:

$${E}_{pot , 0}^{b} = \frac{4}{3} π {R}_{0}^{3} ( {p}_{∞} - {p}_{v} ) ,$$

as described in “The relevance of kinematics for cavitation implosion loads Physics of Fluids”, 31, S. Schenke, T. Melissaris, and T. J. C. van Terwisga, 2019 (Schenke 2019).

In Function 1,

$${E}_{pot , 0}^{b}$$

is the potential energy of the bubble, R₀ is the initial radius of the bubble, (p_{∞}, pᵥ) are ambient pressure and vapor pressure respectively, and this function is valid for an undisturbed spherical bubble. The thermochemically stabilized structure imparts new properties to the fluid, changing its thermal and electrical conductivity, among others, which improves interactions with electronegative structures, for example, cells of a mammalian body.

Further details of the creation of the structured water of this invention, including a system used to create the structured water that can be used in the compositions of this invention, are described herein.

Referring back to FIGS. 5A and 5B, the structural organization of the structured water is shown in these figures. As shown in FIGS. 3 and 4, adjacent water molecules of liquid water at 4° C. are arranged in a hexagonal arrangement, and multiple planes of this hexagonal arrangement of the water molecules are connected via hydrogen bridges to form the three-dimensional helical cage structure shown in FIG. 5A. In this model, the local charge depends on the density of electronegative oxygen atoms. This model explains the changes in electronegativity in the exclusion zone where this configuration occurs, and also explains the changes in the properties such as a 10% higher refractive index than normal water and a higher density than normal water.

FIG. 5B is a top view of the arrangement of the water molecules shown in FIG. 5A. This three-dimensional helical cage structure is created by the cavitation and implosion processes, as described herein.

Homogenization is very important for the proper breaking of the different bonds for the solubilization of molecules. Referring to FIG. 6, the organic salts of the minerals that can be included in the composition of this invention are electronegative in nature, and can organize themselves into a similar arrangement as the arrangement of the water molecules shown in FIGS. 5A and 5B. That is, the atomic size of these elements is such that they can be captured within the hollow lumen created in the three-dimensional helical cage structure of the structured water.

Other properties of the fluid that is formed refer to the electrokinetics obtained from the addition of hydrogen in its gaseous form (H₂) comprising an ionic aqueous solution of nanostructures containing stabilized hydrogen. This gas together with the water molecule, when it touches the surface of a cell wall, modulates a potential of the cell membrane, as well as the electrical properties of the cell membrane. As a result, the ionic aqueous fluid electrokinetically provides regulation of cell membrane potentials and helps with intracellular signal transduction.

Referring back to FIGS. 5A and 5B, the three-dimensional spiral cage structure formed by hydrogen bridging of adjacent molecules based on the energy generated in the cavitation and implosion process creates a channel (hollow lumen), which can trap various components therein. By forming these structures, the water can retain the dissolved hydrogen molecules, minerals, and additives for longer time periods. The stability of the dissolved components is also affected by the interaction of the H₂ bridges with the structured water molecules.

### Mechanism of Water Structuration Process

The formation of the three-dimensional helical cage structure of polygonal water molecules that can be used in the composition of this invention, where adjacent water molecules are connected by hydrogen bridging, based on the energy generated in the cavitation and implosion processes are described herein, including the systems that can be used for implementing the structuration process to produce the structured water.

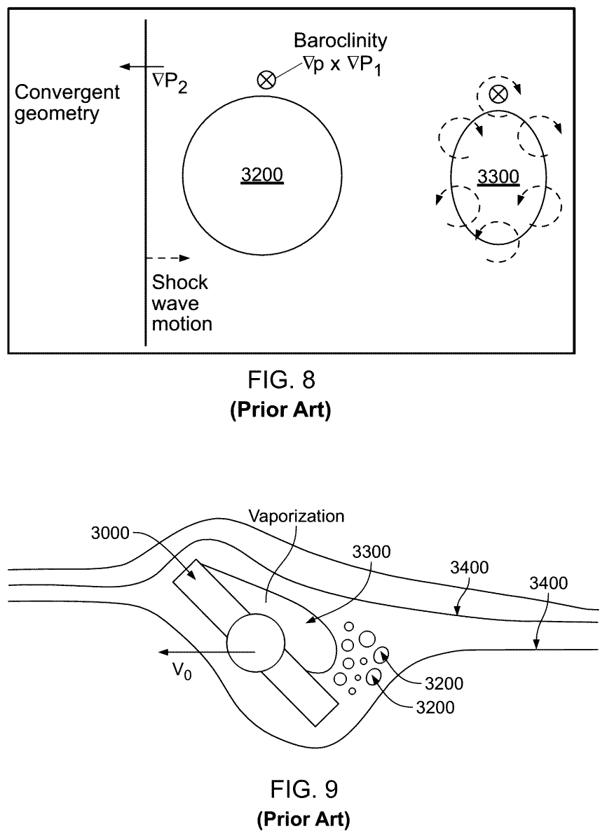

The structuration process can be summarized in three phases, as described here with reference to FIGS. 7A to 7C.

Phase 1. Energy transfer from solid to fluid, where the solid body of high kinetic energy forms a pressure difference on the working fluid that already has a predefined structure due to the contained minerals, and has high kinetic energy. FIG. 7A is an illustration of a high kinetic energy solid, which forms a pressure difference on the working fluid that already has a predefined structure due to the minerals contained in the water. This high kinetic energy solid has high kinetic energy in addition to the internal energy of the fluid. As shown in FIG. 7A, water molecules 100A and mineral atoms 200A, for example, calcium, magnesium, iron, zinc, copper, selenium, and the like, dispersed within the water molecules come into contact with the solid body of high kinetic energy 300A. The high kinetic energy solid 300A is responsible for providing the kinetic energy to the fluid and providing space for the formation of the cavitation and implosion process.

Phase 2. Vacuum pressure, bubble formation, where the solid body of high kinetic energy is removed to create a zone of high vacuum and, due to the thermodynamic properties of water, the water is violently converted from the liquid to the gaseous phase, and this conversion generates a high amount of energy.

Phase 3. The implosion process begins just after the high kinetic energy solid 300A leaves a volume delimited by its geometrical shape, generating a vacuum pressure on the system. In this process, energy is transferred violently and concentrically at various locations because of the creation of a vacuum in the area vacated by the high kinetic energy solid 300A. This process occurs at a local pressure of about 100 MPa and a temperature of about 5000 K, which are generated within the water during the cavitation and implosion processes.

FIGS. 7B and 7C are illustrations of the two zones formed when the removal of the solid body creates a vacuum in collapse zone 400A, and the layer of water molecules 100A closest to the collapse zone 400A changes its phase and becomes a gas, which in turn, raises the temperature of the fluid.

Cavitation describes a phenomenon that occurs inside a liquid when a pressure field is subjected to changes in time and distance. These changes depend on the properties of the liquid which causes the formation of voids, filled with the fluid in its vapor phase, which are then violently compressed, reaching gaseous phases at high pressure and temperature. Due to this process, there is a rapid transfer of energy between a zone where there was previously a vacuum and where the water changes in density.

This phenomenon is caused by a difference in static pressure and vapor pressure of a fluid. When the static pressure of a fluid (pressure of a fluid at rest) is lower than its vapor pressure, small vapor-filled cavities can be present in the fluid. Increasing the pressure on the fluid results in implosion or collapse of these cavities, thereby generating waves of energy emanating from the site of the implosion(s).

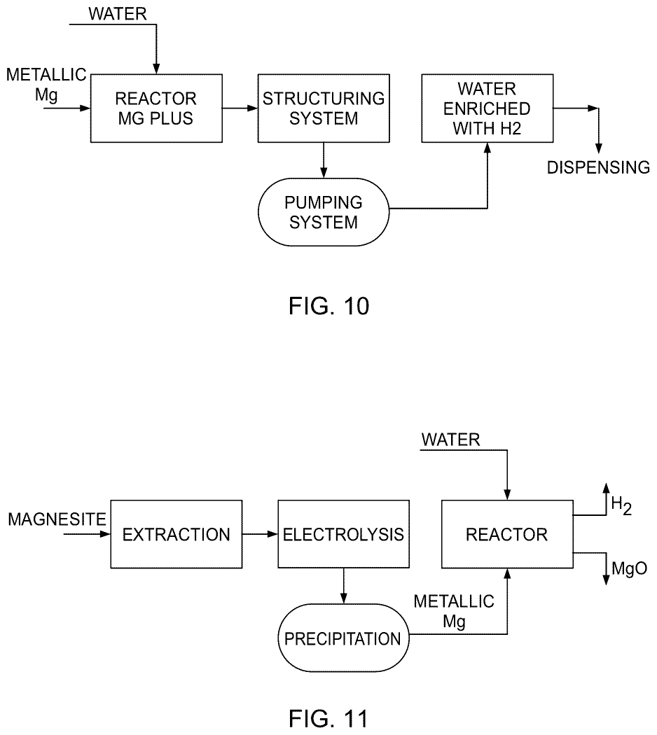

A representative schematic of this process is shown in FIG. 8. In FIG. 8, one cavitation bubble 3200 is shown under normal pressure conditions (prior to exposure to a pressure gradient). When cavitation bubble 3200 is subject to baroclinity (ρ×p1) at a point and converges with an area having a different pressure gradient (p2), the cavitation bubble 3200 is subjected to a shock wave that moves through the fluid due to the difference in the pressure gradients. This causes the cavitation bubble 3200 to implode and form an imploded cavitation bubble 3300, which generates additional energy. Baroclinity, generally denoted by ρ×p, where ρ is a density gradient and p is a pressure gradient of a fluid, is a measure of the misalignment between the density and pressure gradients of a fluid.

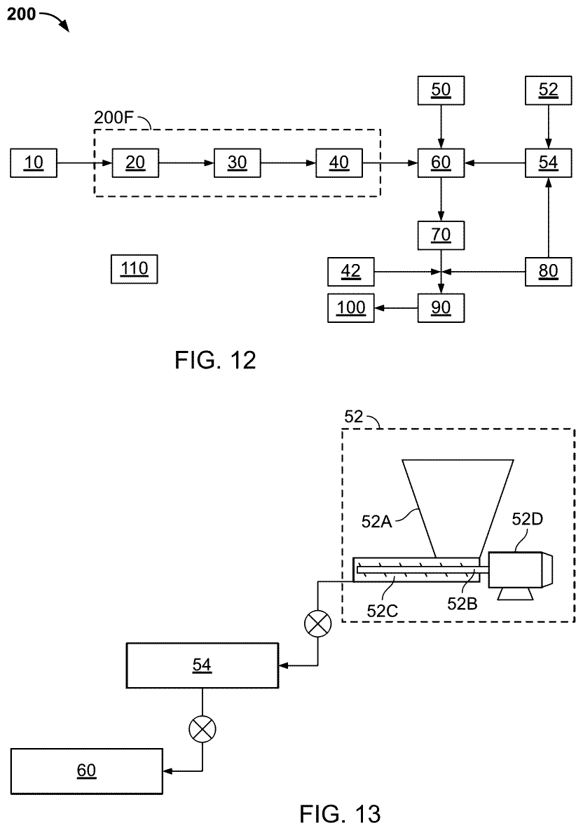

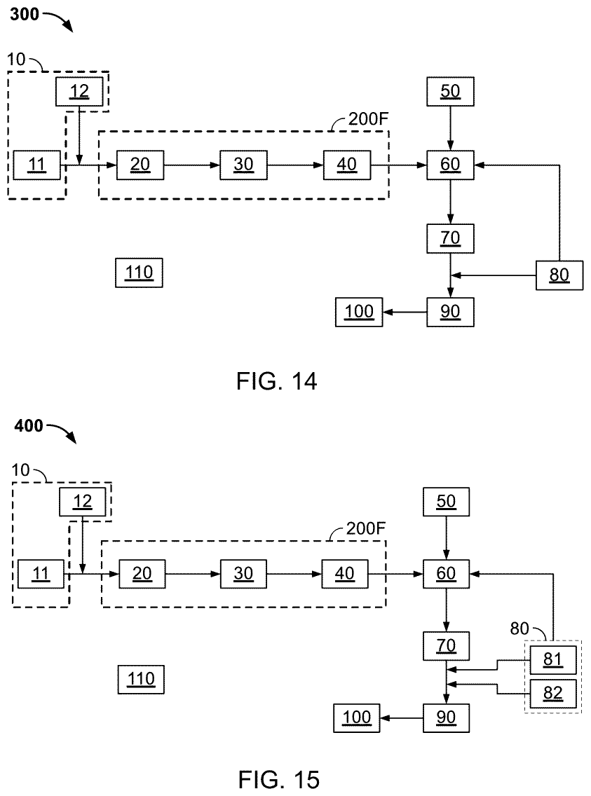

Another schematic representation of this process is shown in FIG. 9. As illustrated in FIG. 10, cavitation bubbles 3200 appear within the fluid when a vortex is generated in a fluid at a velocity V₀ by the action of a rotor (e.g., rotating blade) 3000. As these cavitation bubbles 3200 encounter the pressure differential created by the vortex along isobaric lines 3400, the cavitation bubbles implode into an elliptical-shaped imploded cavitation bubble 3300.

There are various methods for generating the above-described cavitation and implosion processes, including but not limited to: (1) flowing over hydrofoils; (2) supercavitating hydrofoils; (3) flowing over propellers; (4) turbulent cutting flow; (5) using a water inlet cavity; and (6) bubble chambers.

The molecular structures present in structured water, the geometric characteristics of the individual incubation molecules, as well as the groups of molecules, were simulated, and the hydrodynamic impact pressure of the implosion of an individual cavitation bubble was calculated as described herein, based on a qualitative characterization of various parameters, such as the hydrodynamic impact pressure and the impact velocity of a liquid microjet, and the hydrodynamic gravity generated by the cavitation and implosion processes. Most hydrodynamic impacts were in the range of a calculated local pressure of 0.2 GPa to 3 GPa. The calculated temperatures attained in these processes reach more than 5000 K in nanoseconds, which causes the fluid density to change about 1.5 to about 6 times in the zones closest to the implosion.

The water included in the formulation of this application can be obtained from any water source, including but not limited to non-drinkable water that is treated to make it drinkable; a rural or urban water supply network; atmospheric water that is condensed, collected, and used as water source; and the like, but are not limited thereto, and water from any water source can be used.

### Water Structuring System

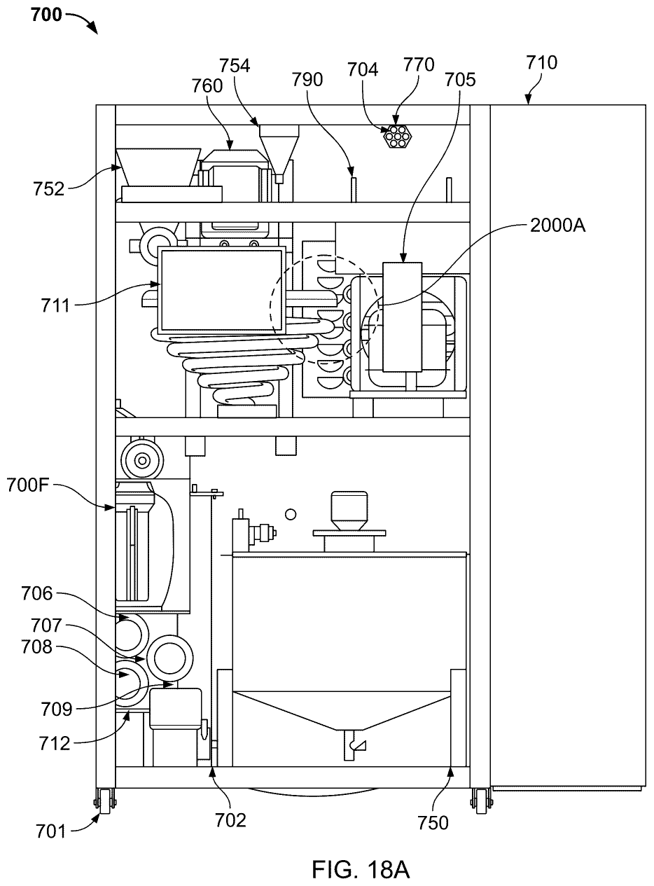

Exemplary embodiments of a water structuring system that can be used to produce the structured water used in this application are illustrated in FIGS. 12-21, and will be described in further detail in this application. The water structuring system includes a vortex generating system to achieve the above-described thermodynamic conditions through the processes of cavitation and implosion. The vortex generating system generates a plurality of microstates producing favorable environments for the generation of hydrogen.

The vortex generates an environment of microstates, which facilitate cavitation and implosion processes resulting in a localized pressure, calculated to be about 0.2 GPa to about 3 GPa and a localized temperature, calculated to be at least 5000 K, in the water that facilitates the formation of structured water. As one example, the vortex can be created by rotating a vortex-generating system at 3600 rpm, which generates an average linear speed of about 50 m/s of the water in the vortex, and an absolute pressure that is less than 2 kPa.

These aforementioned conditions generate pressure and temperature changes in the vortex that make viable the processes of initiation, collision, growth, cavitation cloud, loss of coherence, cavitation cloud growth, collision and implosion. These processes generate temperatures of around 10,000 (K). Consequently, thermolysis of water can occur in the microstates created in the water, and the diameter of these formations or micro-states could reach about 56 μm.

One example of hydrogen production is the reaction of magnesium with water. Recent research has shown that hydrogen can be produced efficiently (with an efficiency of 11% (see, e.g., Shetty et al., A comparative study of hydrogen generation by reaction of ball milled mixture of magnesium powder with two water-soluble salts (NaCl and KCl) in hot water, International Journal of Hydrogen Energy, vol. 45(48), pp. 25890-25899 (2020), ISSN 0360-3199, https://doi.org/10.1016/j.ijhydene.2020.03.156) to 90% (see, e.g., Kushch et al., Hydrogen-generating compositions based on magnesium, International Journal of Hydrogen Energy, vol. 36(1), pp. 1321-1325 (2011), doi:10.1016/j.ijhydene.2010.06.115) using powdered magnesium. Another example is the method described in U.S. Pat. No. 5,494,538 where a magnesium alloy is mixed with minor amounts of one or more metals such as nickel and zinc, which acts as catalysts in the reaction of the magnesium alloy with chlorinated water.

To produce gaseous hydrogen, the amount of granular metallic magnesium used is enough to obtain the maximum solubility of hydrogen in water. The maximum solubility of hydrogen in water ranges from about 1 ppm to about 5 ppm of hydrogen dissolved in water.

By the inclusion of Mg in the water structuring process described herein, the production of hydrogen is increased, while also improving the cavitation and implosion processes. As illustrated in FIG. 10, the metallic Mg and water can be added to a reactor, and then sent to a structuring system.

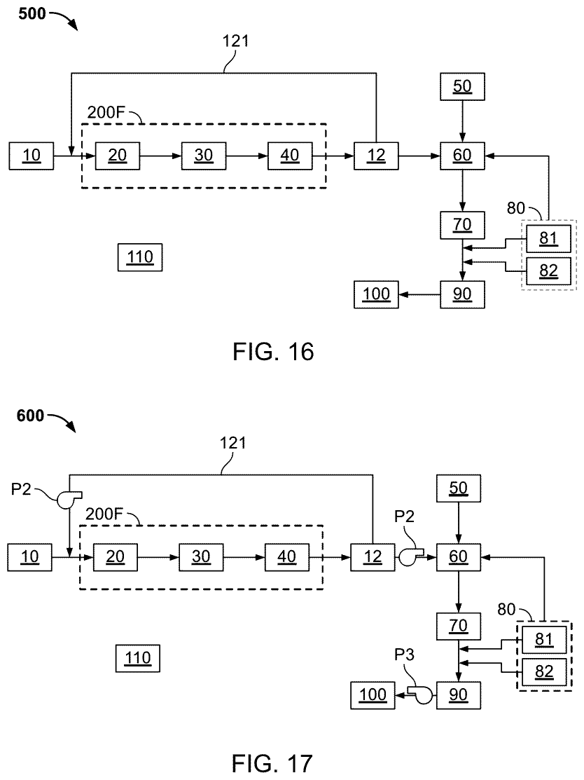

Mg is one example of a mineral that can be used to produce hydrogen in this manner, and also improving cavitation and implosion processes when the process is carried out at appropriate temperature, pressure, time parameters, and the like. As Mg is not found in nature in its pure state, it may be obtained from naturally occurring compounds of magnesium, such as magnesite. Magnesite (generally MgCO₃) is a composition of magnesium salts and other trace elements, such as iron, nickel, manganese, cobalt, and the like. As generally illustrated in FIG. 11, metallic magnesium can be obtained from naturally occurring magnesite using various processes, such as extraction, electrolysis and precipitation, performed in any suitable order, to produced metallic magnesium. The metallic Mg can then be used, as described above, to produce structured water enriched with dissolved hydrogen.

The materials for producing hydrogen are not limited to Mg and magnesite, and any suitable material that reacts with water to produce hydrogen can also be used. Additional examples of such minerals include, but are not limited to alkali and alkaline earth metals such as Na, K, Ca, Sr, Ba, and the like, including any salts thereof.

The particle size of the Mg used can be about 0.01 mm to about 1 mm. The particle size of the Mg can be equal to any integer value or values within this range, including the endpoints of these ranges and any acceptable variance. The particle size of the Mg affects the generation of hydrogen from the reaction of magnesium and water because the geometry of the cluster formed by metallic Mg is dependent on the size of the Mg particle. When the particle size of magnesium that reacts with water is within this range, smaller clusters of Mg are formed, which increase the surface area available for reaction with water and assists in the production of hydrogen bubbles.

Magnesium (Mg) is a very active element and reacts with water at low temperatures to produce magnesium oxide and hydrogen. The reaction can be shifted to producing magnesium hydroxide instead of magnesium oxide by increasing the amount of water. The reactions between magnesium and water are summarized in Equations 1-3:

$${Mg}_{s}^{0} + {H}_{2}^{I} O \to {Mg}^{II} {O}_{( aq )} + {H}_{{2}_{g}}^{0} \qquad Equation 1$$

$${Mg}_{s} + 2 {H}_{2} O ↔ {Mg ( OH )}_{{2}_{( aq )}} + {H}_{{2}_{g}} \qquad Equation 2$$

$$Mg {( OH )}_{2} {↔}^{△} {MgO}_{( s )} + {H}_{2} {O}_{( g )} . \qquad Equation 3$$

The reactions that produce magnesium oxide or magnesium hydroxide are exothermic.

### Designing a Vortex

A two-equation mathematical model that describes the phenomena observed in the water structuring system that can be used to produce structured water for use in this invention is discussed below. A characteristic feature of the two-equation model is a fifth-order nonlinear aerodynamic damping term. Likewise, this model can be used for qualitative analysis, with additional experiments contemplated for quantitative analysis. Based on the two-equation mathematical model, the specific parameters and conditions that create the vortex were designed, as described herein.

The two-equation mathematical model includes Equations A and B:

$$\vec{ω} = ∇ \times \vec{u} \qquad Equation A$$

$$Γ = {\int }_{S} \vec{ω} \cdot \vec{n} dS \qquad Equation B$$

In Equation A, \vec{ω} represents a flow field with velocity distribution u, and \vec{u} represents the velocity distribution of a field. In Equation B, F is defined as a circulation function of a fluid, and S is an arbitrary curved surface. The primary characteristics of the vortices present in a fluid are:

Vorticity at a point in a fluid is a vector. The component of vorticity in a particular direction (\vec{n}) is twice the angular velocity of either of two line segments in the fluid that are mutually orthogonal with \vec{n}. Vorticity is therefore a measure of how fast the fluid rotates.

Just because a flow field is rotating on a large scale, it does not mean that ω in the flux is non-zero (in order to obtain a Γ different from 0, ω should be non-zero at least at one point or in a finite region for a viscous fluid). Even if the current lines of a flow are not curved, the flow itself can be rotational, i.e., ‘vortex lines are material lines’. Vortex lines are lines that are tangential to the local vorticity vector. Vortex tubes are the set of all vortex lines that pass through a finite area. The circulation around a vortex tube is constant, regardless of the shape and location of the contour. As long as a fluid is barotropic, is subject to environmental forces, and only subject to potential corporeal forces, the circulation around any loop of material in the fluid is independent of time. Vorticity is improved by stretching along the axes of rotation of the fluid element. Viscosity causes vorticity to diffuse away from lateral lines. Baroclinity can generate vorticity within a fluid. When the flow is rotational, the vorticity of a fluid element is directly proportional to its density, and the compression of the fluid increases the vorticity.

### Designing Cavitation and Implosion Processes in a Vortex

A model for the onset of cavitation and implosion in a vortex is described here. In this model, a simplified Rayleigh-Plesset single-bubble implosion model is used. The degree of cavitation development is characterized by a non-dimensional parameter known as the cavitation number σ, which is defined by:

$$σ = \frac{{p}_{ref} - {p}_{v}}{\frac{1}{2} ρ {V}^{2}} ,$$

where p_{ref} is the reference pressure of the liquid, pᵥ is the actual pressure of the liquid, ρ is the fluid density, and V is the flow velocity.

The Rayleigh-Plesset equation is a second-order differential equation used to calculate the behavior of the bubble volume as a function of its radius R(t):

$$ρ [ R \ddot{R} + \frac{3}{2} {\dot{R}}^{2} ] = [ {p}_{ν} - {p}_{∞} ( t ) ] + {{p}_{g 0} ( \frac{{R}_{0}}{R} )}^{3 k} - \frac{2 S}{R} - 4 μ \frac{\dot{R}}{R} ,$$

where [pᵥ−p_{∞}(t)] is the difference between the applied pressure and the vapor pressure, and is the driving term of the bubble evolution. The second term of this equation is the contribution of the non-condensable gas, where the constant mass of the gas is assumed to follow a polytropic thermodynamic behavior characterized by a given polytropic coefficient k. S is the surface tension coefficient expressed in N/m or J/m².

Based on the above-described Rayleigh-Plesset model, the specific parameters and conditions that create the vortex, and resulting cavitation and implosion processes were designed.

The design of the implosion system described herein maximizes the implosion phenomenon, maximizes stiffness to prevent the system from reaching its elastic limits and makes it possible to reuse the system, imparts safety, minimizes manufacturing, maintenance, and operating costs, and minimizes weight.

In an exemplary embodiment, to achieve the structured water that can be used in this application, the rotor of the motor is rotated at a rotational speed of about 1800 rpm to about 7000 rpm. The rotational speed can be equal to any integer value or values this range, including the endpoints of these ranges, and any appropriate variances.

The initial pressure inside the structuring chamber during the cavitation and implosion process can be from about 50 kPa to about 105 kPa. The pressure can be equal to any integer value or values within this range, including the endpoints of these ranges, and any appropriate variances. At a pressure within these ranges, the energy of the macrostates of water increases. During the implosion process, the localized pressure of the microstates of water existing in the vicinity of the implosion can reach about 0.2 GPa to about 3 GPa and the localized temperature can be at least 5000 K.

Within these ranges, the system described herein creates the cavitation and implosion processes at the required energy to produce the “structured water” having high hydrogen solubility over time. The structured water and its various components are discussed herein.

The following is a description of the fluid dynamics that form the basis for creating the vortex of this invention to produce the structured water of this invention.

Speed distribution of a Rankine vortex with a central radius a and a maximum circulation Γ is:

$${v}_{θ} = \frac{Γ}{2 π {a}^{2}} r \qquad r \leq a$$

$${v}_{θ} = \frac{Γ}{2 π r} \qquad r > a$$

The total angular momentum per unit length contained within a radius r₀→∞ is:

$$\sum = \frac{1}{2} ρΓ \{ {r}_{0}^{2} - \frac{1}{2} {a}^{2} \}$$

The cavitation vortex is designed such that:

$$r \leq {r}_{i} ( Steam )$$

$$r \geq {r}_{i} ( Liquid ) .$$

(Khojasteh-Manesh et al., “Evaluation of Cavitation Erosion Intensity in a Microscale Nozzle Using Eulerian-Lagrangian Bubble Dynamic Simulation,” J. Fluids Eng., 141(6):061303 (14 pages), June 2019, pub. Online Apr. 4, 2019.)

An exemplary embodiment of a water structuring device that can be used to prepare the structured water that can be used in the compositions of the present disclosure is a water structuring system 200 schematically illustrated in FIG. 12. As shown in FIG. 12, the water structuring system 200 can include a water supply source 10 and a water filtration system 200F. The water filtration system 200F can include a water filter 20, a reverse osmosis filter 30 and a disinfector 40.

In all of the water structuring devices describe herein, the water supply source 10 can be from one or more sources. For example, separately or in combination, the water supply source 10 can be from one or more water supply networks and/or from the moisture in the air which could be condensed, collected, and used as water source. Nevertheless, the water supply source 10 can be any water supply source. One of the advantages of using atmospheric moisture as the water supply source 10 is that it allows the availability of water in absence of traditional sources such as rivers, water supply network, etc.

After obtaining the water from the water supply source 10, the water can be output to the water filter 20. The water filter 20 can include, for example, a sediment filter and/or a filter with any other compound that can aid in the filtration of undesirable components from the water source. Additionally or alternatively, the water filter 20 can include activated carbon. In one embodiment, the reverse osmosis filter 30 can be optional depending on the type or quality of water. For example, the reverse osmosis filter 30 can be used in cases where tap water is used as the water source. In one embodiment, after filtration by the water filter 20, the water can be directed to the reverse osmosis filter 30 and then to the disinfector 40 including an emission of ultraviolet (UV) light. In some embodiments, the disinfector 40 can comprise an ultraviolet (UV) lamp, but is not limited thereto and any suitable disinfection method may be used. Various different types of water filtering devices and disinfecting devices can be used in the water filtration system 200F depending on the quality and type of water source. In some embodiments, there may be no need to include the water filtration system 200F if the quality of water is sufficient for outputting the structured water in accordance with the present disclosure.

Still referring to FIG. 12, the water structuring system 200 can further include a structured water generator 60 coupled, directly or indirectly, to the water filtration system 200F and a mineral supply 50. The water filtration system 200F can purify the water received from the water supply source 10 via the water filter 20, the reverse osmosis filter 30, and the disinfector 40. Then the water can be output to the structured water generator 60 to change the energy structure of the water by agitation and cavitation.

In one embodiment, the structured water generator 60 can receive minerals dispensed from the mineral supply 50 and the purified water discharged from the disinfector 40 or water directly from the water supply source 10. In one embodiment, the mineral supply 50 can add minerals and additives to the water in the structured water generator 60 via a mineral input. The minerals and additives can include, but are not limited to, calcium (Ca), magnesium (Mg), iron (Fe), zinc (Zn), copper (Cu), selenium (Se), one or more amino acids selected from biotin (vitamin B7), folic acid (vitamin B9), thiamine (vitamin B1), riboflavin (vitamin B2), pyridoxine (vitamin B6), cobalamin (vitamin B12), L-alanine, L-valine, L-isoleucine, L-citrulline, L-glutamine, theanine, and the like, and any suitable metabolite of essential amino acids, such as hydroxymethylbutyrate or β-hydroxy β-methylbutyrate, and the like. One or more of these minerals and additives can be in the form of a water soluble salt selected from lactate, sulfate, selenite, halide, nitrate, acetate, hydroxides, and the like, but are not limited thereto, and any suitable anion safe for consumption and/or ingestion can be used. In certain other embodiments, various suitable cations can be used in conjunction with any suitable anion that is safe for consumption and/or ingestion. In certain other embodiments, the mineral is a lactate or a selenite. In certain other embodiments, the mineral is one or more selected from calcium lactate, magnesium lactate, iron lactate, zinc lactate, copper lactate, sodium selenite, and the like. Suitable minerals that can be included in the water composition described herein are not limited, and any mineral or additive that is considered essential for the proper functioning of a human body and/or essential for life and/or considered essential trace elements and/or found in natural mineral water can be used.

In an exemplary embodiment, the water structuring system 200 can comprise a feeder and a discharger (not shown in this figure for clarity of illustration and explanation). The feeder can be any suitable means for feeding a fluid to the water structuring system 200, including but not limited to a pipe, a tube, a valve, a connecting part, and the like, and can be made of any suitable material. The discharger can be any suitable means for discharging a fluid from the water structuring system 200, including but not limited to a pipe, a tube, a valve, a connecting part, and the like, and can be made of any suitable material. One or more of the feeder and the discharger can be formed integrally with the other components in the water structuring system 200 or can be formed separately and connected to the water structuring system 200 through one or more connecting means. Non-limiting examples of connecting means include flanges, adhesives, welding, and the like.

Still referring to FIG. 12, the water structuring system 200 can further include a mineral reactor 52 or a mineral reactor 52 and a mixer 54. For example, the mixer 54 may be a cyclone mixer, but is not limited thereto. Further, the mixer 54 may receive the filtered water from the water filtration system 200F or receive water directly from the water supply source 10, depending on the quality of the water necessary to perform the structuration in accordance with the present disclosure. In one embodiment, the mineral reactor 52 can output H₂, MgO, and water to be input to the mixer 54. In one embodiment, the mixer 54 can receive, simultaneously or sequentially, one or more gases, including but not limited to hydrogen, oxygen, carbon dioxide, and the like, from a gas supply 80.

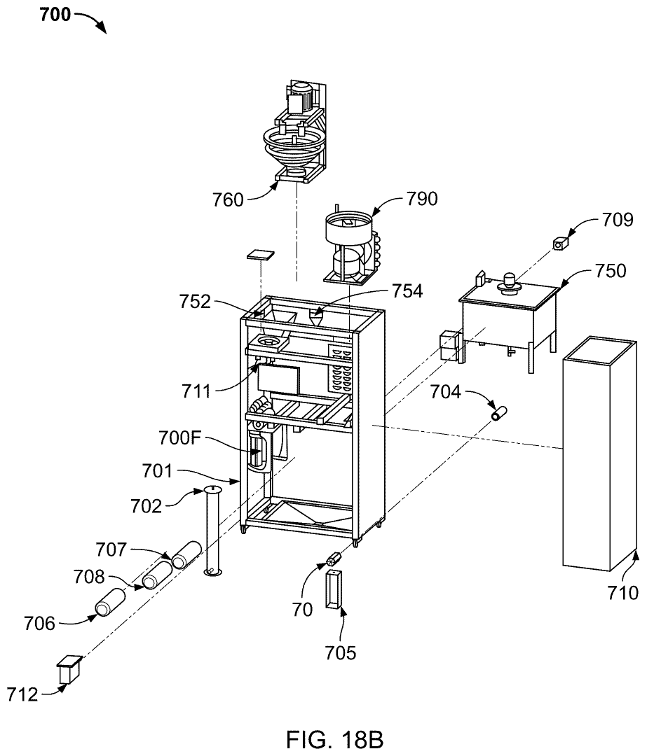

FIG. 13 shows one exemplary arrangement of the mineral reactor 52 and mixer 54 coupled to the structured water generator 60. In this embodiment, the mineral reactor 52 can include a container 52A, a motor 52D, a rotator (or rotary device) 52B, and a housing 52C. The rotator 52B may be a screw-type mixing device (or auger, drill, screw rod, etc.) attached to the motor 52D. Magnesium can be stored in the container 52A. The magnesium stored in the container 52A can be mixed with water by the rotator 52B, as shown in FIG. 13. The reactor (not shown in this figure for clarity of illustration and explanation) can then produce MgO and H₂, which may then be sent to the mixer 54 to be mixed with minerals, additives, and/or additional H₂, in accordance with the present disclosure.

The speed of the mixer 54 (e.g., cyclone mixer) can depend on the desired amount and quality of water being processed in the structured water generator 60. In one embodiment, an average speed of the water in the cyclone mixer can be set at 10 meters/second and the pressure may be 45 psi. However, the speed and the pressure can be varied, based on Bernoulli's principle, depending on the desired amount of MgO and H₂ output from the mineral reactor 52. Referring back to FIG. 12, the water from the mixer 54 can be output to the structured water generator 60 through a feeder described above. In one embodiment, the structured water generator 60 can include one or more blades that can be connected to a shaft that is connected to a speed amplifier. The speed amplifier may include a motor that rotates at high revolutions to generate a vortex in the water, which in turn produces cavitation and implosion, as described earlier in the present disclosure. This phenomenon allows water molecules to reach localized temperatures above about 5,000 degrees Kelvin (K), and depending on the energy generated during the implosion process, the temperature can be about 10,000 K or about 15,000 K, and the like, and, individually, any intervening temperatures. In one embodiment, the structured water generator 60 can comprise a rotating and translating housing structure that translates and rotates a helical-spiral-shaped housing to create the necessary cavitation and controlled implosion processes in the water contained in the helical-spiral-shaped housing. The movement of the rotating and translating housing structure is controlled by any suitable mechanism, including but not limited to actuators, such as a motor that transmits its movement through pulleys to the housing. The housing can be connected channels that direct the flow of the fluid, and lead it to perform rotational and translational movements with a frequency greater than about 300 Hz. These movements lead to a phase change of water into steam that generates the necessary cavitation and controlled implosion processes. The helical/spiral-shaped housing can be, but is not limited to, a tube in the form of a helix or spiral. Additional structural and mechanical details of the structured water generator 60 are later described in more detail.

The onset of cavitation is dependent on the coherent structure of directed flow, which is organized as paired vortex rings. In addition, cavitation/implosion is continuously found in the nucleus of the vortex, indicating a strong correlation between said cavitation/implosion and vortex dynamics. In the initial stage, the stretching of the vortex is the dominant factor, responsible for the growth of the vortex and the elliptical shape of the cavitation bubbles. Inside the water, the cavitation bubbles form an elliptical shape during the implosion process. The elliptical geometry of the imploding cavitation bubbles mirrors the elliptical flow of the fluid, and the cavitation and implosion process is aided by the elliptical geometry of the cavitation bubbles during the implosion process. In comparison, the dilation term could produce enhancement or suppression of local vorticity, depending on the volumetric variation induced by cavitation and, during the implosion stage, the bubble creates baroclinic vorticity and contributes to three-dimensional vorticity. The exposure to cavitation and/or implosion homogenizes the mixture of water, added minerals, additives and dissolved gases. Other processes that provide structuration or homogenize the mixture are ultrasonic mixing or exposure to a vacuum pressure difference, and can form a part of the devices and systems used for the production of structured water than can be used in the compositions provided herein. Based on the periodic functioning of the implosion structure together with the temporal evolution of large eddies, vorticity can be separated into the following nine stages: initiation, collision, growth, cavitation cloud, loss of coherence, cavitation cloud growth, collision, implosion, and water restructuring.

The linear flow rate necessary to start the water restructuring process is in the range of about 30 m/s to 300 m/s. The linear flow rate can be any value or range within this range, including but not limited to the upper and lower limit and any acceptable variance.

Referring back to FIG. 12, the water structuring system 200 can further include a magnetizer 70, a gas supply 80, a cooling system 90, and a dispensing module 100. As discussed above, in the structured water generator 60, minerals and/or additives can be added by the mineral supply 50, and MgO and H₂ may be added by the mineral reactor 52. Additionally or alternatively, the gas supply 80 can provide H₂ to the mixer 54. As described above, the mixer 54 (e.g., cyclone mixer) can mix, in addition to the H₂ from the gas supply 80, H₂ and MgO received from the mineral reactor 52, minerals and/or additives added from the mineral supply 50, and water received form the water filtration system 200F or the water supply source 10. The mixture from the mixer 54 can then be output to the structured water generator 60 to perform the structuration process in accordance with the present disclosure.

After the water leaves the structured water generator 60, the water can be magnetized by the magnetizer 70 with, for example, neodymium magnets, then gases such as oxygen, hydrogen or carbon dioxide can be added, and the structured water can be cooled before being dispensed to a container.

In one embodiment, the magnetizer 70 can comprise any magnetization means that generates a magnetic field preferably strong enough to configure the magnetic field of the water in a desired orientation. Any suitable magnetization means can be used, including but not limited to magnets of metals, such as iron (Fe), cobalt (Co), nickel (Ni), rare earth metals, combinations and alloys thereof; naturally magnetic minerals that are called “calamites” that are composed mostly of iron; and/or electromagnets. In some embodiments, the magnetizer 70 may comprise neodymium magnets. The arrangement of magnets in the magnetizer is not limited, and any suitable arrangement can be used. In some exemplary embodiments, the magnetizer 70 aligns the water molecules by generating an electromagnetic field in a conductive material that produces magnetization by induction. In one embodiment, the cooling system 90 can be arranged to be part of a condenser and/or to maintain a suitable temperature for the structuration of water and/or to cool the final product before being discharged from the water structuring system 200. Further, the cooling system 90 can comprise any suitable means for cooling a fluid, including but not limited an air-cooled system, a water-cooled system, a thermoelectric cooler, an electric cooler, and the like.

Still referring to FIG. 12, in addition to providing H₂ to the mixer 54, the gas supply 80 can provide one or more gases such as oxygen, hydrogen, carbon dioxide, nitrogen, or a combination thereof to the water discharged from the magnetizer 70. For example, CO₂ can be provided to produce carbonation of the water, and oxygen may be added to provide more stable and longer lasting structured water. The gasified water can then be cooled by flowing through the cooling system 90 and dispensed through the dispensing module 100 and into a container (not shown in this figure for clarify of illustration). In one embodiment, the water structuring system 200 can optionally include an additional disinfector 42. The additional disinfector 42 can be similar to the disinfector 40 described above. The disinfector 42 can disinfect or sterilize the water output from the magnetizer 70 before being input to the cooling system 90. All the elements can be controlled and energized by a power supply system (not shown in this figure for clarity of illustration) and a controller 110. Each of components shown in FIG. 12 can be arranged in any order to facilitate the proper functioning of the water structuring device, including being arranged sequentially as shown in FIG. 12.

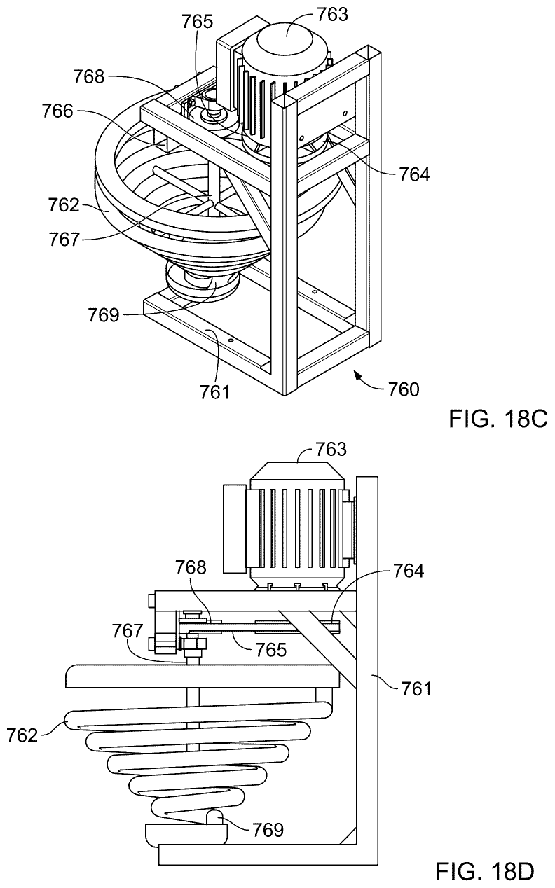

FIG. 14 illustrates an exemplary embodiment of a water structuring system 300. The water structuring system 300 can include the same or similar components as described in the water structuring system 200 shown in FIGS. 12 and 13. The descriptions of the same components shown in FIGS. 12 and 14 are omitted with respect to FIG. 14 for brevity and clarity of explanation. Referring to FIG. 14, the water structuring system 300 can include the water supply source 10 that can include, additionally or alternatively, a direct supply 11 from a water supply network and/or a condensing-collector 12, in which atmospheric moisture is condensed, collected, and stored. In some embodiments, the water structuring system 300 can use only one of the direct supply 11 or the condensing-collector 12. In other embodiments, the water structuring system 300 can use both direct supply 11 and the condensing-collector 12 simultaneously, sequentially, or alternatively together, depending on the availability of water and/or desired amount of water to be processed by the structured water generator 60. The water structuring system 300 including the water supply source 10 shown in FIG. 14 can operate in the similar manner as described in reference to the water structuring system 200 in FIG. 12.

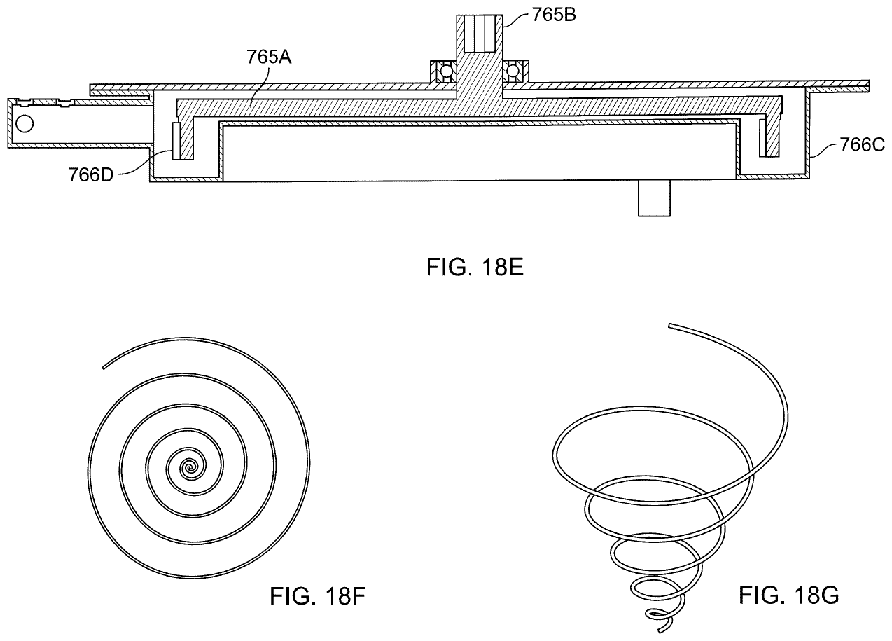

FIG. 15 illustrates a water structuring system 400. The water structuring system 400 can include the same or similar components as described in the water structuring systems 200 and 300 shown in FIGS. 12-14. The description of the same components shown in FIGS. 12-14 are omitted with respect to FIG. 15 for brevity and clarity of explanation. Referring to FIG. 15, the water structuring system 400 can include the gas supply 80 that can include, additionally or alternatively, a first gas supply module 81 and a second gas supply module 82 that can generate or store gases, including but not limited to, oxygen, hydrogen, carbon dioxide and/or nitrogen. The gas supply 80 can include means, structures, or devices for producing (e.g., hydrogen generation cells, Proton Exchange Membrane (PEM) Cells) or separating gases, such as electrolysis or other processes, and means for gas storage, such as cylinders or pressurized tanks. As described above, for example, CO₂ can be provided to produce carbonation of the water (e.g., sparkling water), and oxygen can be added to the water to provide more stable and longer lasting structured water. The water structuring system 400 including the gas supply 80 shown in FIG. 15 can operate in the similar manner as described in reference to the water structuring systems 200 and 300 in FIGS. 12 and 14.

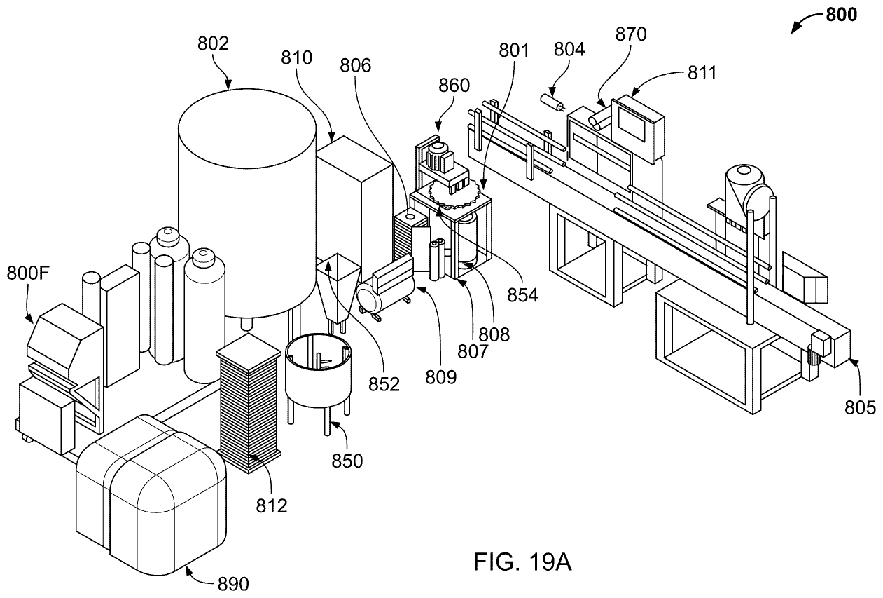

FIG. 16 illustrates a water structuring system 500. The water structuring system 500 can include the same or similar components as describe in the water structuring systems 200-400 shown in FIGS. 12-15. The description of the same components shown in FIGS. 12-15 are omitted with respect to FIG. 16 for brevity and clarity of explanation. The water structuring system 500 can include a condensing-collector 12 coupled, directly or indirectly, between the water filtration system 200F and the structured water generator 60. The condensing-collector 12, which condenses and collects atmospheric moisture, functions as a cooling system that sends condensed water from the air to the input of the water filter 20 through plumbing 121. In one embodiment, the condensing-collector 12 can provide water to the structured water generator 60 without being filtered by the water filtration system 200F. For example, in a desert location where the water in the atmosphere is likely to be clean without or with very little impurities or pollutants, the water condensed from the condensing-collector 12 may be sent directly to the structured water generator 60. The water structuring system 500 including the additional condensing-collector 12 and plumbing 121 can operate in the similar manner as described in reference to the water structuring systems 200-400 in FIGS. 12-15.

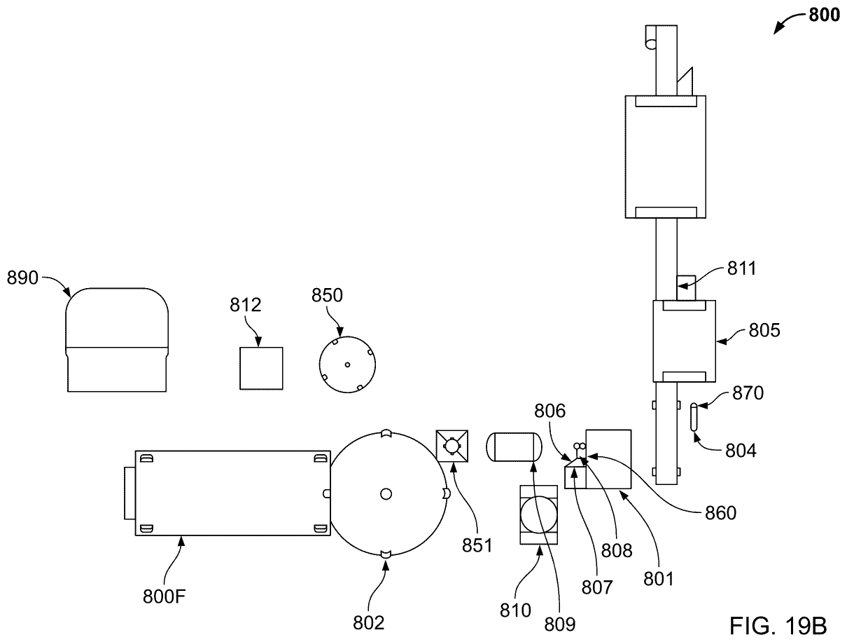

FIG. 17 is a schematic illustration of a water structuring system 600. The water structuring system 600 can include the same or similar components as describe in the water structuring systems 200-500 shown in FIGS. 12-16. The description of the same components shown in FIGS. 12-16 are omitted with respect to FIG. 17 for brevity and clarity of explanation. FIG. 17 shows the locations in the connection pipes where injection pumps P1, P2, and P3 can be located to drive the water under treatment to be discharged. The pumps P1, P2, and P3 can provide suitable pressures to communicate fluid (e.g., water) to and from various components of the water structuring system 600. The arrangements of the injection pumps are not limited thereto, and any suitable arrangement can be used. The water structuring system 600 shown in FIG. 17 may operate in the similar manner as described in reference to the water structuring systems 200-500 in FIGS. 12-16.

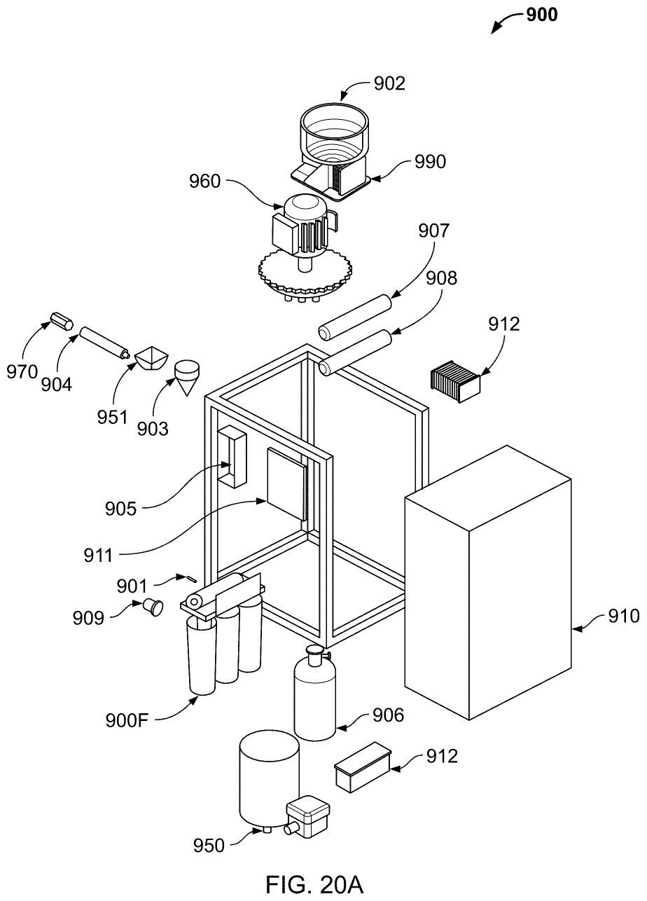

FIGS. 18A and 18B are illustrations of a water structuring system 700, which can incorporate one or more aspects of the water structuring systems 200-600 described in reference to FIGS. 12-17 above. FIG. 18A depicts a front view of the water structuring system 700, and FIG. 18B depicts an exploded view of the water structuring system 700. For the purpose of brevity and clarity of explanation, the water structuring system 700 and its components will be described in reference to FIG. 18A hereinafter. As shown in FIG. 18A, the water structuring system 700 can include a housing 701 and a water supply source 710 arranged adjacent to or coupled, directly or indirectly, to the housing 701. The water supply source 710 can be, for example, an atmospheric humidity collector, which condenses and collects the water contained in atmospheric humidity. In one embodiment, the atmospheric humidity collector can include a cooling system that uses radial or axial fans under thermoelectric coolers, or any other cooling means. The atmospheric humidity collector can alternatively or additionally comprise a fixed-bed steam absorption system that is filled with carbon nanotubes, fullerene and other allotropic forms of carbon that are connected to a helical condenser with a nozzle system that generates a difference in pressure that absorbs steam and improves the process of condensation.

The water structuring system 700 can include, for example, in the housing 701, a fluid storage 702, and a water filtration system 700F. In some embodiments, the water filtration system 700F can include, as disclosed in the foregoing embodiments, the water filter 20, the reverse osmosis filter 30, and/or the disinfector 40. Further, the water filtration system 700F can include, additionally or alternatively, a nanometric filter. Further, the water structuration system can include a mineral reactor (or MgPLUS unit) 752, a structured water generator 760, a mixer 754, and a mineral supply 750. The structured water generator 760 can also include a vortex structuring system (later described in detail in FIGS. 18C-G). The mineral supply 750 can include one or more pumps to maintain the homogeneity of the desired mineral mixture in the water.

The water collected by the water supply source 710 (e.g., water supply source 10 and/or condensing-collector 12) can be fed, for example, to the fluid storage 702 in the housing 701, as shown in FIG. 18A. The collected or stored water in the fluid storage 702 can then be sent to the water filtration system 700F (e.g., the water filter 20, the reverse osmosis filter 30, the disinfector 40, and/or a nanometric filter) to filter or purify the water. The structured water generator 760 can also receive minerals dispensed from the mineral supply 750. The mineral supply 750 can add minerals and/or additives to the water in the structured water generator 760 via a mineral input. The trace elements can include, but are not limited to, calcium (Ca), magnesium (Mg), iron (Fe), zinc (Zn), copper (Cu), selenium (Se), one or more amino acids selected from biotin (vitamin B7), folic acid (vitamin B9), thiamine (vitamin B1), riboflavin (vitamin B2), pyridoxine (vitamin B6), cobalamin (vitamin B12), L-alanine, L-valine, L-isoleucine, L-citrulline, L-glutamine, theanine, and the like, and any suitable metabolite of essential amino acids, such as hydroxymethylbutyrate or β-hydroxy β-methylbutyrate, and the like. The minerals and additives added to the system can be any one or more suitable minerals and additives, including but not limited to, any minerals and additives described herein.

The water structuring system 700 can comprise a feeder and a discharger (omitted in FIG. 18A for clarity of illustration and explanation). The feeder can be any suitable means for feeding a fluid to the water structuring system 700, including but not limited to a pipe, a tube, a valve, a connecting part, and the like, and can be made of any suitable material. The discharger can be any suitable means for discharging a fluid from the water structuring system 700, including but not limited to a pipe, a tube, a valve, a connecting part, and the like, and can be made of any suitable material. One or more of the feeder and the discharger can be formed integrally with the other components in the water structuring system 700 or can be formed separately and connected to the water structuring system 700 through one or more connecting means. Non-limiting examples of connecting means include flanges, adhesives, welding, and the like.

Still referring to FIG. 18A, the filtered water from the water filtration system 700F can be provided to the mineral reactor 752 and the mixer 754. The mineral reactor 752 can produce H₂ and MgO to be sent to the structured water generator 760. As illustrated in FIG. 13, the mineral reactor 752 can include the container 52A, the motor 52D, the rotator 52B, and a housing 52C. The rotator 52B can be a screw-type mixing device (or auger, drill, screw rod, etc.) attached to the motor 52D. Magnesium can be stored in the container 52A. The magnesium stored in the container 52A can be mixed with water by the rotator 52B. The reactor (not shown in the figure for clarity of illustration and explanation) can then produce MgO and H₂, which can then be sent to the mixer 754 to be mixed with minerals, additives and/or additional H₂. The speed of the mixer 754 (e.g., cyclone mixer) can depend on the desired amount and quality of the water being processed in the structured water generator 760. In one embodiment, an average speed of the water in the mixer 754 (e.g., cyclone mixer) can be set at 10 meters/second and the pressure may be 45 psi. However, the speed and the pressure may be varied, based on the Bernoulli's principle and the desired amount of MgO and H₂ output from the mineral reactor 752.

The amount of minerals and/or additives added to the mineral reactor 752 and the minerals and/or additives received by the structured water generator 760 from the mineral supply 750 can vary. One or more minerals and/or additives received by the structured water generator 760 from the mineral supply 750 can assist in inducing cavitation and/or agitation in the structured water generator 760.

The structuring process of the structured water generator 760 is described further in detail hereinafter. The water from the mixer 754 can be provided to the structured water generator 760 to change the energy structure of the water, by means of agitation and then exposed to cavitation, and subsequent implosion. As disclosed above, the mineral and additives can be added to the structured water generator 760 from the mineral supply 750. The addition of minerals, such as magnesium, improves the generation and/or retention of desired gases (e.g., hydrogen, oxygen, carbon dioxide, etc.) in the water.

The structured water generator 760 can be any device or means that can provoke sufficient cavitation, implosion and/or agitation in the water to induce structuration of the water. The structured water generator 760 can include, as described above, various input and output means to introduce apt-to-drink water, minerals and additives and elements that induce cavitation and/or agitation such as spinning device coupled to the structured water generator 760.

The structured water generator 760 can comprise a rotating and translating device (i.e. a device that provides structuration to water) that translates and rotates a helical-spiral-shaped container containing water to generate the necessary cavitation and controlled implosion processes for structuring the water. FIGS. 18C-E show an exemplary implementation for the structured water generator 760 including the rotating and translating mechanism. As shown in FIG. 18C, the structured water generator 760 can include a housing (or a bracket or frame) 761. In or on the housing 761, the structured water generator 760 can include a motor 763, a first wheel 764, a second wheel 768, and a belt 765 that is fitted into the groove of each of the first wheel 764 and the second wheel 768, as shown in FIGS. 18C and 18D. The combination of the first wheel 764, the second wheel 768, and the belt 765 can be referred to as a rotation generator. The first wheel 764 and the second wheel 768 can have different diameters to multiply the speed or torque generated by the pully system. For example, the first wheel 764 can be a 6-inch wheel, and the second wheel may be a 4-inch wheel, but are not limited thereto, and any suitable size and number of wheels can be used in the rotation generator.

The motor 763 is coupled to the first wheel 764 that rotates to provide sufficient rotational and translational movements of the structured water generator 760 at a frequency greater than 300 Hz. These movements lead to a phase change from water into steam that generates the necessary cavitation and controlled implosion processes of the present disclosure. In one embodiment, the motor 763 can include, as shown in FIG. 18E, a rotation element 765A in a housing 766C of the motor 763. The rotation element 765A can include one or more magnets 766D that facilitates the rotation of the rotation element 765A. The motor 763 can include one or more coils for generating a magnetic field to generate rotational force against the one or more magnets 766D. The motor 763 can include a shaft 765B that can be connected to the first wheel 764 to rotate of the first wheel 764 for facilitating the water structuration process.

Referring back to FIG. 18C, the structured water generator 760 can comprise a conical-shaped (or spiral-shaped) container (or tank) 762 having an input opening 766, which can be coupled, directly or indirectly, to the mixer 754, structured water generator 760, mineral supply 750, and/or water supply source 710 to receive desired fluid and/or minerals to facilitate structuration of water in accordance with one or more aspects of the present disclosure. The conical-shaped container 762 can be, for example, a helical-spiral-shaped tube (i.e. a tube that has the form of a helical spiral). The structured water generator 760 can comprise an output opening 769 to output structured water from the conical-shaped container 762. The conical-shaped container 762 can have a capacity of 15 to 50 liters, but is not limited to this capacity. The structured water generator 760 can include a shaft 767, which can include rods (or blades) that are connected to one or more internal surfaces of the conical-shaped container 762, as shown in FIG. 18C. The shaft 767 can be connected to the motor 763 that rotates at high revolutions to generate a vortex, which allows the water to produce the phenomenon of cavitation and consequently an implosion of each bubble generated in the conical-shaped container 762.

As shown in FIGS. 18C and 18D, the one or more screws and nuts, as well as other suitable fastening elements, can be utilized to securely arrange the components of the structured water generator 760 in the housing 761. That is, the components of the structured water generator 760 shown in FIGS. 18C and 18D can be attached or coupled to the housing 761 in the manner sufficient to support translational and rotational movements of the conical-shaped container 762 at high speeds. The translational and rotational movement will be described with reference to FIG. 18D. The translational and rotational movements of the conical-shaped container 762 allows the water molecules in the conical-shaped container 762 to reach localized temperatures above 5000 K. In some embodiments, the temperatures could triple depending on the energy generated from the translation and rotational movements. The onset of cavitation exhibits a great dependence on the coherent structure of directed flow, which is organized as paired (or concentric) vortex rings shown in FIGS. 18F and 18G. In addition, cavitation/implosion can continuously occur in the nucleus of the vortex, indicating a strong correlation between said cavitation/implosion and vortex dynamics. In the initial stage, the stretching of the vortex can be the dominant factor, responsible for the growth of the vortex and the elliptical shape of the cavitation ring. In comparison, the dilation term could produce enhancement or suppression of local vorticity, depending on the volumetric variation induced by cavitation and, during the implosion stage, the bubbles create baroclinic vorticity and contribute to three-dimensional vorticity. The exposure to cavitation and/or implosion homogenize the mix. In one embodiment, structuration or homogenization of the mix can be achieved through ultrasonic mixing or exposure to a vacuum pressure difference. The periodic functioning of the implosion structure together with the temporal evolution of large eddies, vorticity can be separated into, for example, the following 9 stages: initiation, collision, growth, cavitation cloud, loss of coherence, cavitation cloud growth, collision, implosion and water restructuring. In one embodiment, the linear flow rate necessary to start the water restructuring process may be in the range between 30 m/s to 300 m/s.

Still referring to FIG. 18A, the water structuring system 700 can include a magnetizer 770 and a dispensing module 705. The magnetizer 770 can include, for example, any means or device that generates a magnetic field sufficient to configure the magnetic field of the water in a desired manner. For example, the magnetizer 770 may include, but not limited thereto, neodymium magnets or other magnetization means, such as one, or a combination, of the following: magnets of metals such as iron (Fe), cobalt (Co), and/or nickel (Ni); naturally magnetic minerals that are called “calamites” that are composed mostly of iron; and/or electromagnets. The magnets or other magnetization means can be arranged in the water structuring system 700 in any configuration in accordance with the desired design or functionality of water structuring system. Additionally or alternatively, the magnetizer 770 can align the water molecules by generating an electromagnetic field in a conductive material that produces magnetization by induction. After the water leaves the structured water generator 760, the water can be magnetized by the magnetizer 770, then gases such as oxygen, hydrogen or carbon dioxide can be added if desired, before being cooled and finally dispensed to a container.

Still referring to FIG. 18A, the water structuring system 700 can include, in the housing 701, a gas supply including, for example, at least one of a H₂ storage 706, an 02 storage 707, and a CO₂ storage 708, a hydrogen generation cell 712, or a combination thereof. The water structuring system 700 can also include a cooling system 790, a main control system 711, a compressor 709, and a UV filter 704.

In one embodiment, the gas supply (e.g., H₂ storage 706, O₂ storage 707, CO₂ storage 708, and/or hydrogen generation cell 712) can add one or more gasses (e.g., oxygen, hydrogen, carbon dioxide, nitrogen, or a combination thereof) to the water that can be treated by the structured water generator 760. In one embodiment, the gas supply can include means or structure (e.g., hydrogen generation cell 712) to perform separation of water into gaseous oxygen and hydrogen using electrolysis or other processes, and means or structure for gas storage, such as cylinders or pressurized tanks. In one embodiment, before the gas supply adds one or more gasses to the treated water, the UV filter 704 can disinfect or sterilize the structured water processed from the structured water generator 760. Additionally, the water can be cooled by the cooling system 790 before being dispensed by the dispensing module 705. The cooling system 790 can also be used to cool the water supplied to the structured water generator 760 to a temperature of 4° C.

As described above, FIG. 18B depicts an exploded view of the water structuring system 700. FIG. 18B illustrates one exemplary arrangement of the components of the water structuring system 700. Of course, other arrangements of the components are possible to facilitate the desired operation of the water structuring system 700. Since the water structuring system 700 shown in FIG. 18B includes the same or similar components as describe in the water structuring system 700 shown in FIG. 18A, the descriptions of the same components shown in FIG. 18A are omitted accordingly for brevity and clarity of explanation. In embodiments, the water structuring system 700 of FIGS. 18A and 18B can comprise various feeders and/or dischargers coupled to various components of the water structuring system 700 shown in FIG. 18B, to facilitate operation of the water structuring system 700. The feeders can be any suitable means for providing fluids, minerals, and/or other materials necessary to facilitate operation of the water structuring system 700, including but not limited to a pipe, a tube, a valve, a connecting part, and the like, and can be made of any suitable material. The dischargers can be any suitable means for discharging fluids, minerals, and/or other materials necessary to facilitate operation of the water structuring system 700, including but not limited to a pipe, a tube, a valve, a connecting part, and the like, and can be made of any suitable material. One or more of the feeder and the discharger can be formed integrally with the water structuring system 700 or can be formed separately and connected to the water structuring system through a connecting means. Non-limiting examples of connecting means include flanges, adhesives, welding, and the like.

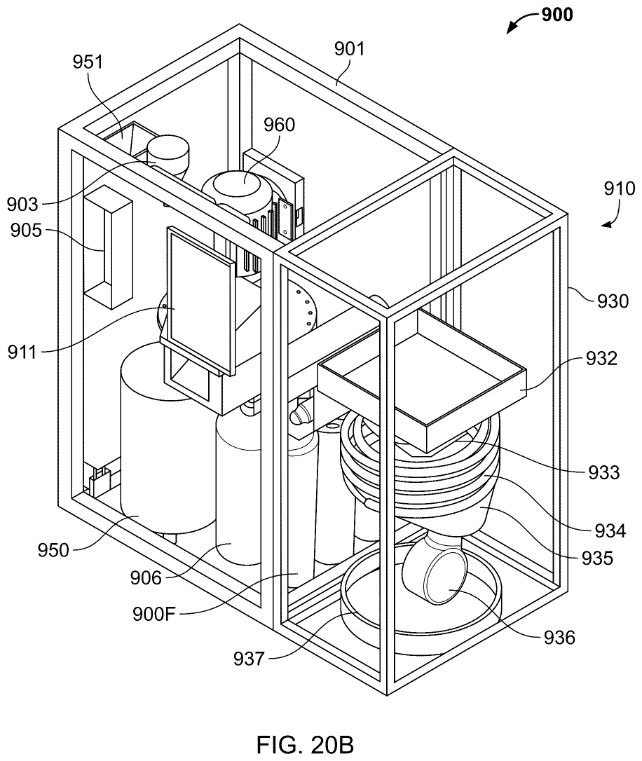

FIGS. 19A and 19B are illustrations of a large-scale water structuring system 800. In one embodiment, the water structuring system 800 can include a water filtration system 800F, a housing 801, a fluid storage 802, a UV filter 804, a dispenser 805, an H₂ storage 806, an O₂ storage 807, CO₂ storage 808, a hydrogen generating 809, a water supply source 810, a main control system 811, a hydrogen generation cell 812, a mineral supply 850, a mineral reactor (or MgPLUS unit) 852, a mixer 854, a structured water generator 860, a magnetizer 870, and a cooling system 890. Although the size, shape, and placement (or arrangement) of the components shown in FIGS. 19A and 19B can be different from the components of the water structuring system 700 shown in FIGS. 18A-E, the components of the water structuring systems 700 and 800 are scalable and modifiable to yield the same structured water. As such, the detailed descriptions of each of the components of the water structuring system 800 are omitted with respect to FIGS. 19A and 19B for brevity. FIG. 19A is a perspective view of the large-scale water structuring system 800, and FIG. 19B is a top down view of the large-scale water structuring system 800.

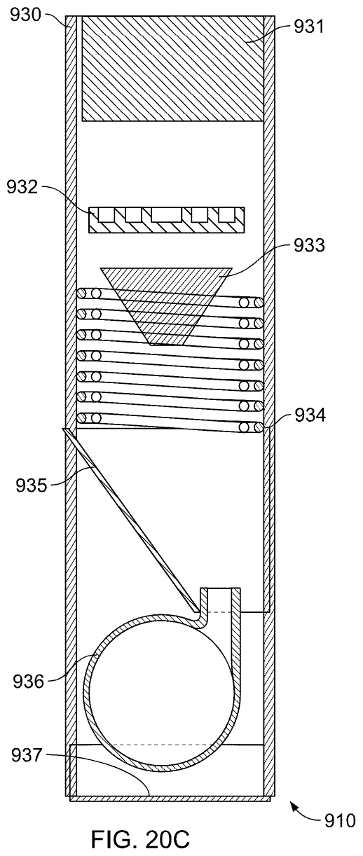

FIGS. 20A and 20B are illustrations of a compact version of a water structuring system 900. In one embodiment, the water structuring system 900 can include a water filtration system 900F, a housing 901, a fluid storage 902, a UV filter 904, a dispenser 905, an H₂ storage 906, an O₂ storage 907, CO₂ storage 908, a water supply source 910, a main control system 911, a hydrogen generation cell 912, a mineral supply 950, a mineral reactor (or MgPLUS unit) 903, a mixer 951, a structured water generator 960, a magnetizer 970, and a cooling system 990. Although the size, shape, and placement (or arrangement) of the components shown in FIGS. 20A and 20B cab be different from the components of the water structuring systems 700 and 800 shown in FIGS. 18A-E and 19A-B, the components of the water structuring systems 700-900 are scalable and modifiable to yield the same structured water. As such, the detailed descriptions of each of the components of the water structuring system 900 are omitted with respect to FIG. 20A for brevity. FIG. 20A is an exploded view of the compact water structuring system 900, and FIG. 20B is a perspective view of the water structuring system 900.

FIGS. 20B and 20C illustrate the components of the water structuring system 900 and the water supply source 910. The components in the water supply source 910 can be incorporated into the water supply sources of the systems 200-800 in FIGS. 12-19B. In one embodiment, the water supply source 910 can be a condensation and extraction system. When the water supply comes from moisture in the environment, the water supply source 910 can comprise an optimized condensation system with an extraction system that allows capturing water from the atmosphere by two main elements, a condensation system and an extraction system.

The water supply source 910 can include a condensation system housing 930, a cooling system 932, and a steam absorber 933, and a condenser 934. In one embodiment, the cooling system 932 can be a semiconductor-based electronic component that functions as a small heat pump based on the Peltier effect. By applying a low DC electrical voltage to it, one side of the device will be cooled while the other side will be heated simultaneously. This device is used to improve the coefficient of performance (COP) of the module and improves the heat transfer rate (i.e. increases the ability of heat transfer). The steam absorber 933 can be a fixed-bed steam absorber, which absorbs steam, that is filled with carbon nanotubes, fullerene and other allotropic forms of carbon that are connected to the condenser 934. The condenser 934 can be a helical-spiral-shaped housing, and the condenser 934 can be connected to a nozzle system 935, which improves the process of condensation. In one embodiment when a helical-spiral-shaped housing is used as the condenser 934, the cooling system 932 (e.g., thermoelectric cooler) can alternatively be attached to the condenser 934 (e.g., helical-spiral-shaped housing) for allowing a better arrangement of the thermoelectric cells. The condenser 934 (e.g., helical-spiral-shaped housing) can be located above an air flow that is injected by an extractor for condensation. The water supply source 910 may also include an air extractor 936, and a storage container 937.

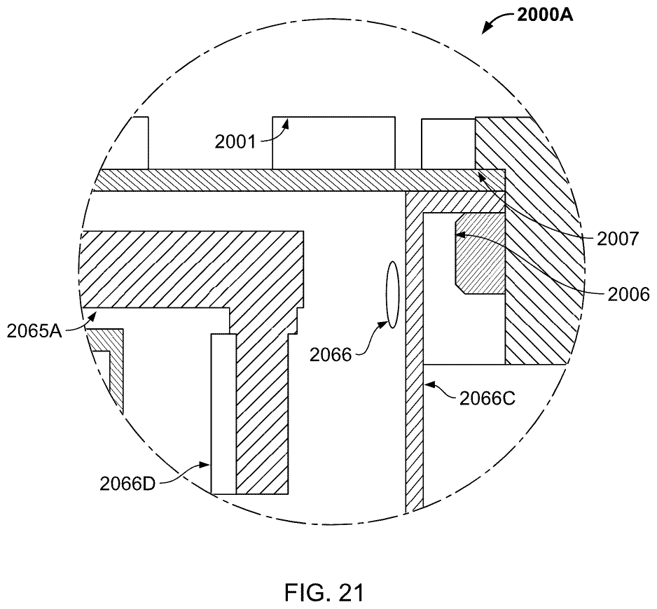

FIG. 21 is a cutaway view of area 2000A of the water structuring system 700 as shown in FIG. 18A. FIG. 21 shows the attachment of the structured water generator 760 to the water structuring system 700, and illustrates the movement of the various parts, for example, the conical-shaped (or spiral-shaped) container (or tank) 762, during the cavitation process. For example, as shown in FIG. 21, the water structuring system 700 includes a primary fastening system 2001, a rotation element 2065A, an input opening 2066, one or more magnets 2066D (high energy solid), a housing 2066C for the rotation element 2065A, a secondary fastening system 2006, and a sealer 2007. In an exemplary embodiment, the primary fastening system 2001 is a mechanical temporary fixing device that, by means of a torsional force, is responsible for joining the housing 2066C and the sealer 2007. The rotation element 2065A guides the rotational movement of the one or more magnets 2066D by conveying torque and force. The input opening 2066 includes a hole for injecting fluid, minerals and/or additives into the apparatus. The input opening 2066 is not limited, and any suitable input for materials to be added to the water structuring system can be used.

The one or more magnets 2066D (high energy solid) are responsible for displacing fluid inside the structured water generator 760 at high speeds, which generates turbulent flow and current trajectories that can be derived in circular and helical forms, thereby generating an empty area where high pressures and high temperatures can be found inside the structured water generator 760. The one or more magnets 2066D (high energy solid) along with the sealer 2007 are also responsible for avoiding leaks produced at high pressures, which prevents depressurization and ensures a hermetic system within the water structuring system 700, including the structured water generator 760, while also providing rigidity to the system. The secondary fastening system 2006 is a mechanical element that allows for the containment and fixing of removable elements.

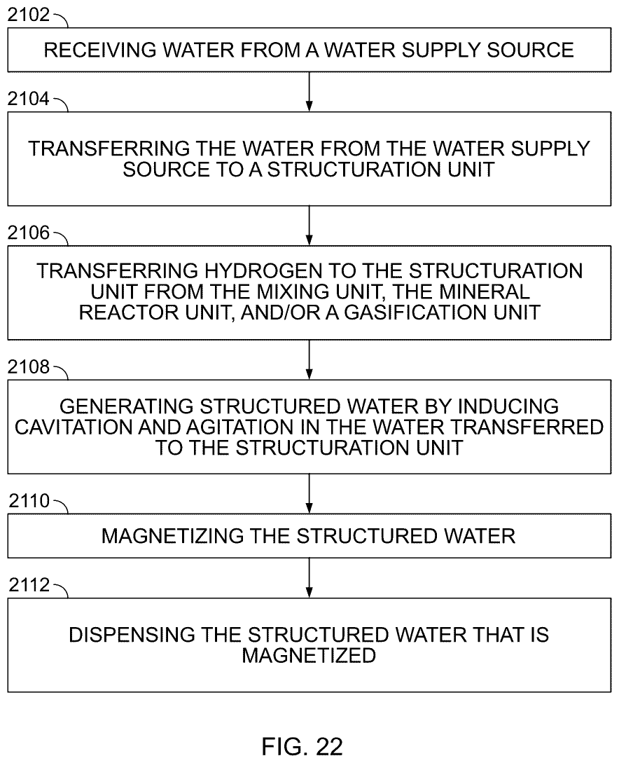

FIG. 22 depicts a flowchart of an exemplary method 2100 for producing structured water by a water structuring system. The water structuring system performing the method 2100 can utilize any of the systems and components described above in reference to FIGS. 12-21 to produced structured water. At step 2102, a water structuring system device can receive water via a water supply source. In one embodiment, the water supply source can include a condenser, which may generate water from humidity in the atmosphere. In one embodiment, the water received via the water supply source can be filtered by a water filtration system. At step 2104, the water from the water supply source can be transferred to a structured water generator. The water also can be transferred to a mixer and/or a mineral reactor (e.g., MgPLUS unit). The water transferred to the structured water generator, mixer, and/or the mineral reactor can be from the water supply source and/or from the water filtration system. The mineral reactor can generate MgO and H₂ from the received water. At step 2106, the mixer, the mineral reactor, and/or a gas supply can transfer hydrogen to the structured water generator. The mixer can mix MgO and H₂ received from the mineral reactor with the filtered water received from the water filtration system. The mixer can mix any suitable water with H₂ received from a gas supply. The mixer can mix, with any suitable water, MgO and H₂ received from the mineral reactor and H₂ received from a gas supply. A mineral supply can transfer one or more minerals and/or additives to the structured water generator. For example, the minerals and/or additives can be the same as disclosed in the foregoing disclosure.

Still referring to FIG. 22, at step 2108, the structured water generator can generate structured water by inducing cavitation and implosion in the water transferred to the structured water generator. In one embodiment, the water can be transferred to the structured water generator from the water received from the water supply source, the water filtration system and/or a fluid mixture can be received from the mixer. In one embodiment, the cavitation and agitation/implosion can be generated by a vortex generator of the structured water generator. The vortex generator can be configured to rotate at, for example, 3600 rpm to generate an average linear speed of water of about 30 m/s to about 60 m/s, and preferably 50 m/s. Further, the vortex generator can be configured to maintain an internal pressure that is less than 2 kPa absolute. In another embodiment, the vortex generator can be configured to generate an average linear speed of water at 10 m/s, and can be configured to maintain an internal pressure of 45 psi. In one embodiment, the structured water generator can structurize the filtered water received from the water filtration system and/or the fluid mixture received from the mixer. Alternatively, the structured water generator can structurize only the fluid mixture received from the mixer. In one embodiment, structured water generator can structurize any suitable water received from the water supply source, water filtration system, and/or the mixer with one or more minerals received from the mineral supply or with one or more additives.

At step 2110, a magnetizer can magnetize the structured water output from the structured water generator. In one embodiment, the magnetizer may generate a magnetic field to rearrange the molecules in the structured water to be close to each other to yield a better tasting and longer lasting structured water. In one embodiment, a UV filter can disinfect or sterilize the structured water that is magnetized and/or the gas supply can add one or more gases to the structured water that is magnetized. For example, the one or more gases can include oxygen, hydrogen, carbon dioxide, nitrogen, or a combination thereof. In one embodiment, a cooling system can cool the structured water that is magnetized to a desired temperature. At step 2112, a dispenser may dispense the structured water that is magnetized to a container.

In one embodiment, a main control system can automatically or manually facilitate the water structuration method in accordance with the present disclosure, including method 2100. For example, the water structuring systems described herein can include one or more user interfaces. The user interfaces can be a display, knob, button, lever, touchscreen, and/or any other suitable input terminal configured to receive user inputs for initiating the water structuration process of the present disclosure. The main control system can be connected, directly or indirectly, to the components of the water structuring system of the present disclosure to facilitate electrical and mechanical control and/or actuation of the components of the water structuring system for performing the structuring and dispensing of the structured water. The main control system can include one or more processors and instructions executable by the one or more processors that may be stored on a non-transitory computer-readable medium. Whenever a computer and/or processor (e.g., automated or manual control of the water structuring system by a control system) implemented method is described in this disclosure, this disclosure shall also be understood as describing a non-transitory computer-readable medium storing instructions that, when executed by one or more processors, configure and/or cause the one or more processors to perform the computer-implemented method. Examples of non-transitory computer-readable medium include RAM, ROM, solid-state storage media (e.g., solid state drives), optical storage media (e.g., optical discs), and magnetic storage media (e.g., hard disk drives). A non-transitory computer-readable medium may be part of the memory of a computer system or separate from any computer system.

In some methods, the aqueous composition containing fullerene can be prepared by including the fullerene and the micelle-forming agent with the minerals or additives in the water delivered to the structured water generator. The structured water generator disperses the fullerene and micelle-forming agent, resulting in micelle-surrounded fullerene nanoparticles. The micelle-surrounded fullerene nanoparticles can be located within the hollow lumen of the three-dimensional helical cage structure of the polygonal water molecules of the resulting structured water. Additional hydrogen gas can be included.

In some methods, the aqueous composition containing fullerene can be prepared by a method that includes preparation of structured water as described above containing minerals and other additives, preparation and solubilization of the fullerene, and the mixing of these two components together under a method using a vortex that results in cavitation and implosion in the water to produce the final formulation. The micelle-surrounded fullerene nanoparticles can be located within the hollow lumen of the three-dimensional helical cage structure of the polygonal water molecules of the structured water. Additional hydrogen gas can be included.

In some methods, the aqueous composition containing fullerene can be prepared by a process that includes the preparation and solubilization of the fullerene, the preparation of an aqueous solution containing minerals and other additives, and the mixing of these two components together under a structuring method using a vortex that results cavitation and implosion forming structured water. The micelle-surrounded fullerene nanoparticles can be located within the hollow lumen of the three-dimensional helical cage structure of the polygonal water molecules of the structured water. Additional hydrogen gas can be included.

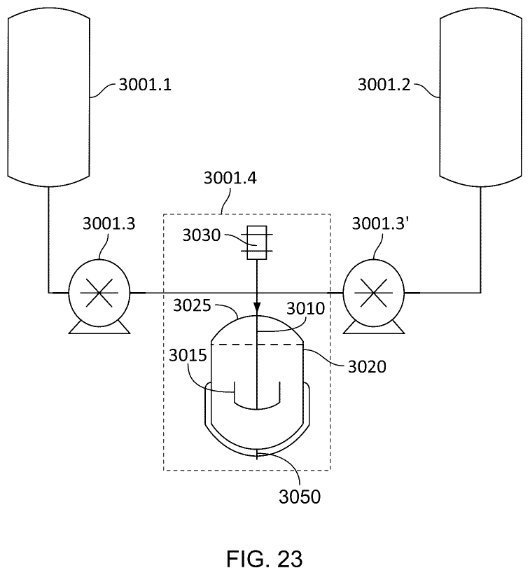

An exemplary process for preparing the micelle-surrounded fullerene is shown in FIG. 23.

The fullerene powder is weighed and deposited in the stainless steel container 3001.1 shown in FIG. 23. The amount of fullerene dispensed to device 3001.4 from container 3001.1 is an amount that results in a final concentration of fullerene in the resulting aqueous formulation from about 0.1 to 30,000 mg/L. In some formulations, the amount dispensed is an amount that results in a final concentration of fullerene in the resulting aqueous formulation from about 1 to 3,000 mg/L.

This fullerene powder goes to the device 3001.4. Subsequently a selected amount within the range described above of olive oil or coconut oil or essential oil or any combination thereof is added to stainless steel container 3001.1, and by means of the positive displacement pump 3001.3, the material is deposited in vessel 3020 of device 3001.4.

Device 3001.4 is a vortex-producing device that includes a rotating shaft 3010 with end blades 3015 in the form of a mechanical stirrer that is placed in a vessel 3020. The rotating shaft 3010 with end blades 3015 is made of stainless steel. The end blades 3015 can have a size and shape configured to produce the desired vortex, and can be selected and arranged depending on the size of vessel 3020. Any size and configuration of end blades on a rotating shaft known in the art can be used.

This vessel 3020 is U-shaped, to improve the mixing properties of the product. Vessel 3020 has a lid 3025 that can be opened and/or removed. The lid 3025 is a top cover that fits on the vessel 3020 to prevent the raw material in vessel 3020 from escaping during mixing and processing, and maintains the desired pressure and temperature conditions within vessel 3020. Lid 3025 can include an access port that allows the addition of other different types of additional raw materials during the process. The vessel 3020 can be made of food-grade plastic or stainless steel, and can be configured for variable capacity. For example, the vessel can be configured to have a capacity ranging from 0.1 to 5 liters. Vessel 3020 can include one or more raw material inlets. For example, the vessel can contain two raw material inlets. The one or more inlets can be in a sidewall of vessel 3020, or can be located in lid 3025. The vessel can contain one or more outlets. In some configurations, the vessel can contain one outlet. In the embodiment shown in FIG. 23, the outlet 3050 is located at the bottom of vessel 3020.

In container 3001.2 of FIG. 23, a surfactant, such as polysorbate 80 is added, previously weighed in the selected quantity within the range described above. Water in an amount that is from 10% to 30% of the total weight of the final composition, and any other elements of the formulation, also can be added to container 3001.2. Subsequently, by means of the positive displacement pump 3001.3′, the material in container 3001.2 is transferred to vessel 3020 of device 3001.4. Alternatively, container 3001.2 can contain bulk surfactant, such as polysorbate 80, and the positive displacement pump 3001.3′ can be activated to dispense the targeted amount of surfactant into vessel 3020 of device 3001.4. Then the water and any other elements of the formulation can be added into vessel 3020 of device 3001.4.

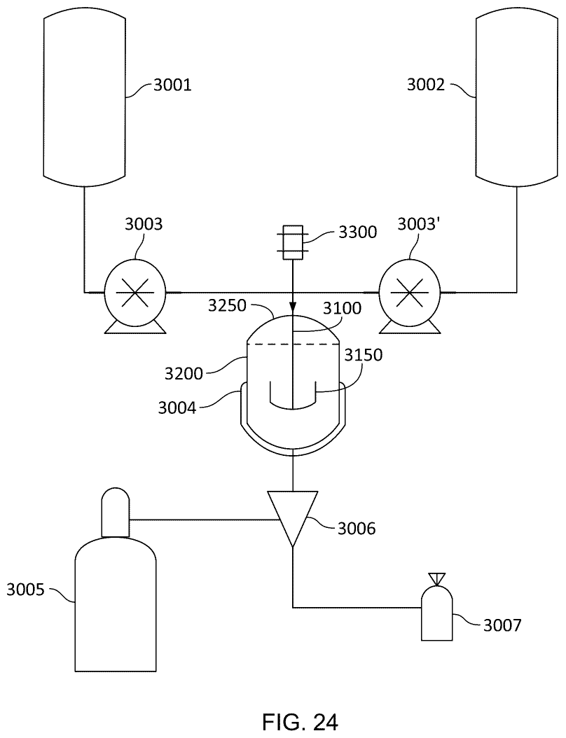

The mechanical stirrer or rotator 3030 engages with the rotating shaft 3010 with end blades 3015 to rotate rotating shaft 3010 to move the material inside the vessel 3020. Sufficient energy is provided by the mechanical stirrer or rotator 3030 to rotating shaft 3010 with end blades 3015 to homogeneously mix the components. Then sufficient energy is provided by the mechanical stirrer or rotator 3030 to rotating shaft 3010 with end blades 3015 to create a large amount of non-laminar vortices in the water, which generate small vapor bubbles in the water, resulting in cavitation and implosion processes. This creates a highly energetic environment in the liquid, which can generate extreme localized conditions of pressure and temperature in the water, up to 1 GPa and 5000 K, respectively in areas in the vicinity of the cavitation and implosion. The range of energy supplied to the fluid can vary from about 1,000 kJ to 60,000 kJ. The output of this process is structured water with water-soluble fullerene, which is added to vessel 3001 of FIG. 24.

The typical size of the micelle-fullerene in the output from the process can range from 5 to 1000 nm in diameter, or from 5 to 100 nm in diameter. Formation of the micelle-fullerene can be done at a pH of from about 4.5 to 9 and a temperature of from 1 to 35° C. In some embodiments, the process is conducted at room temperature, while some frictional heating of the material within vessel 3020 can occur.

Referring to FIG. 24, the water-soluble fullerene diluted in a micellar fluid in vessel 3001, and water in vessel 3002, are introduced into vessel 3004 by activation of the positive displacement pumps 3003 and 3003′. In some embodiments, the water is purified water. In some embodiments, the water is distilled or deionized water. In some embodiments, the water in vessel 3002 is structured water produced by the method described in U.S. patent application Ser. No. 18/100,562, filed by BEST PLANET SCIENCE LLC on Jan. 23, 2023, and in U.S. patent application Ser. No. 18/100,563, filed by BEST PLANET SCIENCE LLC on Jan. 23, 2023, the entire disclosure of each of which is incorporated herein by reference, and which is described in detail above with respect to the water structuring systems.

Device 3004 is a vortex-producing device similar to the vortex-producing device 3001.4 of FIG. 23. Device 3004 includes a rotating shaft 3100 with end blades 3150 in the form of a mechanical stirrer that is placed in a vessel 3200. This vessel 3200 is U-shaped, to improve the mixing properties of the product. Vessel 3200 has a lid 3250 that can be opened and/or removed. The lid 3250 is a top cover that fits on the vessel 3200 to prevent the raw material in vessel 3200 from escaping during mixing and processing, and maintains the desired pressure and temperature conditions within vessel 3200. Lid 3250 can include an access port that allows the addition of other different types of additional raw materials during the process. The vessel 2200 can be made of food-grade plastic or stainless steel, and can be configured for variable capacity. For example, the vessel can be configured to have a capacity ranging from 0.1 to 5 liters, but is not limited to these capacities. Vessel 3200 can include one or more raw material inlets. For example, the vessel can contain two raw material inlets. The one or more inlets can be in a sidewall of vessel 3200, or can be located in lid 3250. The vessel can contain one or more outlets. In some configurations, the vessel can contain one outlet. In the embodiment shown in FIG. 24, the outlet is located at the bottom of vessel 3200, and is connected to mixing device 3006, which can introduce hydrogen into the material discharged from the outlet of vessel 3200.

A mechanical stirrer or rotator 3300 engages with the rotating shaft 3100 with end blades 3150 to rotate rotating shaft 3100 to move the material inside the vessel 3200. Sufficient energy is provided by the mechanical stirrer or rotator 3300 to rotating shaft 3100 with end blades 3150 to homogeneously mix the components. Then sufficient energy is provided by the mechanical stirrer or rotator 3300 to rotating shaft 3100 with end blades 3150 to create a large amount of non-laminar vortices in the water, which generate small vapor bubbles in the water, resulting in cavitation and implosion processes. This creates a highly energetic environment in the liquid, which can generate localized extreme conditions of pressure and temperature in the water, up to 1 GPa and 5000 K, respectively in the vicinity of the cavitation and implosion. The range of energy supplied to the fluid can vary, but typically is more than the energy used to produce the water-soluble fullerenes in the micellar fluid. For example, the range of energy supplied to the fluid can be from 2,000 kJ to 60,000 kJ. The output of this process is output the mixing device 3006.

Mixing device 3006 is connected to a hydrogen regulated cylinder 3005. Mixing device 3006 performs mixing of the fluid containing fullerene, minerals and hydrogen with additional hydrogen. Hydrogen gas is added to mixing device 3006 from the hydrogen regulated cylinder 3005 at a pressure in a range varying from about 0.1 to 5 bar. After a period of time from about 1 minute to 1 hour, the hydrogen-containing fluid in mixing device 3006 is transferred to system 3007 from which it can be dispensed. The aqueous formulation dispensed from system 3007 is suitable for human consumption as dispensed, or can be packed into a unit dosage form for administration to a subject in need thereof.

The combination of micelle-fullerene and hydrogen and structured water, and optionally minerals, can affect bioavailability in a positive way due to their geometry and magnetic charge, enhancing their ability to interact with other molecules in the body.

The geometry of the fullerene molecule can influence its solubility, which makes it highly insoluble in water, however when processed as described above to yield the aqueous formulations described herein, the ability of the fullerene to interact with different media and in its capacity to bind to proteins and other cellular components is increased. In addition, the magnetic charge associated with molecules can influence their ability to bind to specific receptors and participate in biochemical reactions. Molecules with positive or negative charges can interact with proteins and other molecules that have opposite charges, which can affect their distribution and bioavailability in the body. The stability of the fullerene in the structured water described herein is protected due to the matrix that surrounds it, and can exhibit improved viscosity properties, which can reduce its rupture by mechanical elements.

### Hydrogen

The compositions provided herein include hydrogen. When water is structured, it increases the capacity to retain dissolved hydrogen and change its diamagnetic properties compared to traditional water. Structured water increases retention capacity of hydrogen compared with traditional drinking water. The dissolved hydrogen can be produced during the water structuring process. For example, one example of hydrogen production is the reaction of magnesium with water, as discussed herein. By the inclusion of Mg in the water structuring process described herein, the production of hydrogen is increased, while also improving the cavitation and implosion processes. The materials for producing hydrogen are not limited to Mg and magnesite, and any suitable material that reacts with water to produce hydrogen can also be used. Additional examples of such minerals include, but are not limited to alkali and alkaline earth metals. In addition, hydrogen gas can be included in the water, with or without the Mg in order to provide hydrogen. Further, after the structure water exits the structured water generator, hydrogen gas can be added. In addition, as shown in FIG. 24, hydrogen gas can be added to the composition after preparation of the composition, such as by use of mixing device 3006.

In the aqueous compositions provided herein, the amount of hydrogen gas present can be in a range of 1 to 50 ppm. The amount of hydrogen gas present can be in a range of 1 to 40 ppm, or 1 to 30 ppm, or 1 to 20 ppm, or 1 to 10 ppm.

The dissolved hydrogen gas in the structured water can be contained within the lumen of the structured water, resulting in nanostructures containing stabilized hydrogen. This gas together with the water molecule, when it touches the surface of a cell wall, can modulate a potential of the cell membrane, as well as the electrical properties of the cell membrane. As a result, the ionic aqueous fluid electrokinetically provides regulation of cell membrane potentials and helps with intracellular signal transduction, which can trigger uptake of the nanoparticles.

### Minerals and Additional Elements

The aqueous formulation provided herein can also include minerals and/or additional elements. The minerals can include one or more selected from calcium (Ca), magnesium (Mg), iron (Fe), zinc (Zn), copper (Cu), and selenium (Se), but are not limited thereto, and any suitable mineral can be included in any suitable amount. One or more of these minerals can be in the form of a water soluble salt selected from lactate, sulfate, selenite, halide, nitrate, acetate, hydroxides, and the like, but are not limited thereto, and any suitable anion safe for consumption and/or ingestion can be used. In certain other embodiments, various suitable cations can be used in conjunction with any suitable anion that is safe for consumption and/or ingestion. In certain other embodiments, the macro- and/or micro-nutrient is a lactate or a selenite. In certain other embodiments, the mineral is one or more selected from calcium lactate, magnesium lactate, iron lactate, zinc lactate, copper lactate, sodium selenite, zinc sulfate, copper (II) sulfate pentahydrate, and the like. Suitable minerals that can be included in the water composition described herein are not limited, and any mineral that is considered essential for the proper functioning of a human body and/or essential for life and/or considered essential trace elements and/or found in natural mineral water can be used provided the added minerals do not significantly affect the taste of the final composition.

The concentration of calcium salt, present in certain embodiments of the composition of this invention as calcium lactate but not limited thereto, can be about 100 mg/L to about 8200 mg/L. The dissolved calcium concentration can be equal to any integer value or values within this range, including the end-points of these ranges and any acceptable variance. The concentration of magnesium salt, present in certain embodiments of the composition of this invention as magnesium lactate but not limited thereto, can be about 40 mg/L to about 5800 mg/L. The dissolved magnesium concentration can be equal to any integer value or values within this range, including the end-points of these ranges and any acceptable variance. The concentration of iron salt, present in certain embodiments of the composition of this invention as iron lactate but not limited thereto, can be about 1 mg/L to about 40 mg/L. The dissolved iron concentration can be equal to any integer value or values within this range, including the end-points of these ranges and any acceptable variance. The concentration of zinc salt, present in certain embodiments of the composition of this invention as zinc lactate but not limited thereto, can be about 1 mg/L to about 20 mg/L. The dissolved zinc concentration can be equal to any integer value or values within this range, including the end-points of these ranges and any acceptable variance. The concentration of copper salt, present in certain embodiments of the composition of this invention as copper lactate but not limited thereto, can be about 0.01 mg/L to about 2.0 mg/L. The dissolved copper concentration can be equal to any integer value or values within this range, including the end-points of these ranges and any acceptable variance. The concentration of selenium salt, present in certain embodiments of the composition of this invention as sodium selenite but not limited thereto, can be about 0.001 mg/L to about 0.5 mg/L. The dissolved selenium concentration can be equal to any integer value or values within this range, including the end-points of these ranges and any acceptable variance. The aforementioned concentrations of elements not only provide health benefits, but also increase the retention of hydrogen in the composition of this invention.

The additional elements can provide energy, improve cardiovascular activity and replenish nutrients lost during strenuous activity, exertion and/or physical training. The additional elements can be any suitable element, compound or composition that provides the discussed properties, including but not limited to one or more branched-chain amino acids, creatinine, β-alanine, L-camitine, β-hydroxy β-methylbutyric acid (HMB), thiamine, casein, glucosamine, collagen, hyaluronic acid, cysteine, methionine, arginine, aspartic acid, glutamic acid, glycine, histidine, phenylalanine, proline, threonine, lysine, tyrosine, Vitamin A, Vitamin C, Vitamin D, Vitamin E, Vitamin K, isoflavones, chenopodin or 11S-type globulin, 2S albumin, choline, protease, lipase, amylase, lactase, sunflower lecithin, 7-keto dehydroepiandrosterone (DHEA), diindolylmethane, arbutin, ursolic acid, and tannic acid.

### Additives

The compositions also can include other additives, such as a sweetener, one or more flavoring agents, a flavor enhancer, or combinations thereof. The sweetener can be a natural sugar or a sugar substitute. The sweetener can be sucrose. The sweetener can be selected from the group consisting of sucrose, stevia, steviol glycosides, monk fruit extract, sucralose, isomalt, aspartame, saccharin, acesulfame potassium, neotame, advantame, lactitol, xylitol, high-fructose corn syrup, and combinations thereof.

Flavoring agents can be a single chemical or a blend of chemicals whose primary purpose is to provide all or part of the particular flavor or effect desired. The flavoring agent can be in the form of oils or extracts, and can be water soluble or solubilized in water with a surfactant. The flavoring agent can be an essential oil. The flavoring agent can be acidic, basic, neutral or a salt. The flavoring agents can include a natural flavor, a natural fruit flavor, menthol, cinnamon, spearmint, an artificial flavor, an artificial fruit flavor, or combinations thereof. The flavoring agent can be any one or a combination of the flavoring agents known in the art, such as those available from International Flavor and Fragrances, Inc., or listed in Table 2 of U.S. Patent Application Publication 20060292189 (Xia et al., 2006), incorporated herein by reference. The flavor enhancer can be anhydrous citric acid, lactic acid, sodium gluconate, sodium gluconolactate, ethyl lactate, sodium lactate, sodium acid sulfate, malic acid, and combinations thereof. The sweetener and/or the flavoring agent and/or the flavor enhancer, when present, can be present in an amount from about 0.0001 to 20 wt %. The additive can be added with the water added to the micelle-fullerene prior to processing in vortex device 3004.

### D. METHODS

Provided is a method of treating cancer in a subject. Patient treatment options often are limited, and any additional treatment options or methods for improving existing treatments by providing improved therapeutic benefit would enhance treatment opportunities for patients with cancer. Thus, there is a continuing need for new treatment options for patients with cancer. Provided herein is a method of treating cancer in a subject, the method including administering to the subject a therapeutically effective amount of an aqueous composition including fullerene disclosed herein, which can be administered alone or in combination with a pharmaceutically acceptable carrier. The cancer can be selected from among breast, prostate, liver, colorectal, gastrointestinal, pancreatic, skin, thyroid, cervical, lymphoid, hematopoietic, bladder, lung, renal, ovarian, uterine, and head or neck cancer. Specific examples include medulloblastoma; chronic lymphocytic leukemia; adenocarcinoma of the breast, colon, esophagus, liver, lung, pancreas, prostate, stomach, rectum, or uterus; infiltrating ductal breast carcinoma; and cerebellar tumor. The cancer can be skin cancer or a basal cell carcinoma.

The method of treating cancer in a subject includes administering to a subject in need thereof a composition provided herein that includes structured water, fullerene, hydrogen, and a micelle-forming agent. The administration of the composition provided herein can be alone or in conjunction with second therapeutic treatment, such as a chemotherapy, a radiotherapy, an immunotherapy, or a combination thereof. The treatment can be continued for a period of weeks, and can be repeated if needed depending on the type, severity, and stage of cancer in the patient being treated.

The fullerene can be or include an unmodified C60 or C70 fullerene. The fullerene can be or include a C60 or C70 fullerene modified to include one or more than one hydrophilic group on a surface of the fullerene. The hydrophilic group can be a hydroxyl group, a carboxy group, a carboxylic acid group, an alcohol group, an amine group, or a sulfonate group. The fullerene can be a polyhydroxylated fullerene known as fullerenol. The fullerene can be or include a C60 or C70 fullerene modified to be complexed with a water-soluble polymer. The water-soluble polymer can be selected from the group consisting of a polyethylene glycol, a dextran, a polyvinyl alcohol, a polysaccharide, and a protein.

The fullerene can be included in an amount of 1 mg/L to 30,000 mg/L. In some formulations, the amount of fullerene is 1 mg/L to 20,000 mg/L, or 1 mg/L to 10,000 mg/L, or 1 mg/L to 5,000 mg/L, or 1 mg/L to 1,000 mg/L, or 1 mg/L to 500 mg/L, or 1 mg/L to 250 mg/L, or 1 mg/L to 100 mg/L, or 100 mg/L to 1,000 mg/L, or 200 mg/L to 800 mg/L.

The amount of hydrogen gas present in the composition can be in a range of 1 to 50 ppm. In some formulations, the amount of hydrogen gas in the formulation is 5 to 50 ppm, or 10 to 50 ppm, or 1 to 10 ppm.

The micelle-forming agent in the composition can include a surfactant, a liposoluble agent, an oil, or a combination thereof. The oil can be an essential oil, an olive oil, a coconut oil, or a combination thereof. The micelle-forming agent can include a nonionic surfactant. The nonionic surfactant can be selected from the group consisting of a sorbitan ester, a polyalkoxylated sorbitan fatty acid ester, a polyoxyethylene ether, a polysorbate, and a combination thereof. The polysorbate can be selected from the group consisting of polysorbate 20, polysorbate 40, polysorbate 60, and polysorbate 80. The amount of the micelle-forming agent in the composition can be 50 mg/L to 800 g/L.

The composition also can include minerals, a flavoring agent, a flavor enhancer, a sweetener, or any combination thereof. Any mineral known to have a therapeutic effect can be included in the formulation. The minerals can be one or more of calcium (Ca), magnesium (Mg), iron (Fe), zinc (Zn), copper (Cu), and selenium (Se). The minerals can be present in an amount from 0.005 mg/L to 800 mg/L. When present, the sweetener can be selected from the group consisting of sucrose, stevia, steviol glycosides, monk fruit extract, sucralose, isomalt, aspartame, saccharin, acesulfame potassium, neotame, advantame, lactitol, xylitol, high-fructose corn syrup, and combinations thereof. When present, a flavor enhancer can be selected from among a citric acid, a lactic acid, a sodium gluconate, a sodium gluconolactate, an ethyl lactate, a sodium lactate, a sodium acid sulfate, malic acid, and combinations thereof. In the formulations, the sweetener and/or the flavoring agent and/or the flavor enhancer, when present, can be present in an amount from about 0.0001 to 20 wt %.

The composition can be administered in a dose that delivers an amount of fullerene in the range of 10 to 100 mg per dose. Exemplary doses include 10 mg per day, 20 mg per day, 30 mg per day, 40 mg per day, 50 mg per day, 60 mg per day, 70 mg per day, 80 mg per day, 90 mg per day, or 100 mg per day. The dose can be delivered once per day, or divided to be delivered in two or three aliquots during the day. In some embodiments, the full dose is delivered once per day.

The dose can be delivered orally, or via any nonparenteral route of administration. Orally ingested fullerenes can reach the target tumor through processes of intestinal absorption and systemic distribution, where they can be transported through the bloodstream and selectively accumulate in the tumor tissue due to increased permeability and enhanced retention of nanoparticles in the tumor blood vessels. The presence of hydrogen in the formulation containing fullerenes affects the formulation efficacy in various ways, as it can alter their physical and chemical properties such as solubility, stability, and drug-loading capacity due to interactions with water, cells, tissues, and fullerenes, affecting their ability to penetrate the tumor microenvironment and exert specific therapeutic effects. The formulation can impact the bioavailability and biocompatibility of fullerenes, which can impact their effectiveness as therapeutic agents in cancer treatment.

The dosing schedule can vary, depending on the subject and the cancer being treated. General examples include daily administration, twice weekly administration, or weekly administration. Specific examples of the present invention include administering daily for 6 days followed by one day of no treatment, or daily for 5 days followed by two days of no treatment, or daily for 4 days followed by one day of no treatment, or daily for 3 days followed by one day of no treatment, or daily for 2 days followed by one day of no treatment, or administered every other day. Administration can be for one week or multiple weeks, typically for at least two weeks. In some cases, administration can continue for 2 to 10 weeks, or 1 to 6 months. A drug holiday of 1 to 5 days between dosing can be included. A boost dose administration (a dosage include 10 to 400% more fullerene than the normal daily dosage) can be administered at the beginning of each dosing schedule, or the end of each dosing schedule, or after 2, 3, 4, or more seeks of treatment. For example, a daily dosage can include 20 mg fullerene, and a boost dosage can include 80 mg fullerene.

The dose can be modified depending on the type of patient and patient tolerance to the administration of the formulation. For example, lower doses can be prepared for pediatric or elderly patients, and modified doses can be prepared for obese patients or patients with coexisting disease states (e.g., kidney disease, diabetes, or cirrhosis).

Also provided is a method of administering photodynamic therapy to a subject in need thereof, the method comprising (i) selecting a site of the subject where the photodynamic therapy is to be administered; (ii) administering to the subject an aqueous composition including fullerene disclosed herein, which can be administered alone or in combination with a pharmaceutically acceptable carrier; and (iii) exposing the site to natural or artificial light for a period of time effective to achieve a therapeutic effect. The subject can have a disorder selected from among a cancer, psoriasis, acne, actinic keratosis, eczema, seborrhea and hyperkeratinosis. The cancer can be selected from among breast, prostate, liver, colorectal, gastrointestinal, pancreatic, skin, thyroid, cervical, lymphoid, hematopoietic, bladder, lung, renal, ovarian, uterine, and head or neck cancer. The cancer can be skin cancer or a basal cell carcinoma. The cancer can be selected from among an adenocarcinoma, a medulloblastoma, chronic lymphocytic leukemia, and a cerebellar tumor.

Also provided is a method for producing a drug delivery vehicle, comprising complexing a therapeutic agent to the fullerene of the fullerene-containing compositions provided herein, to form the drug delivery vehicle. The fullerene core is very hydrophobic, and can form an inclusion cage around a water insoluble active ingredient. Functional groups can be attached to the core to further modify the behavior of fullerene molecule. The water-soluble fullerenes provided herein are capable of carrying drugs and genes for the cellular delivery. Derivatized fullerene can cross the cell membrane.

### E. EXAMPLES

The principles of the present invention, as well as certain exemplary features and embodiments thereof, will now be described by reference to the following non-limiting examples. However, it will be apparent to those skilled in the art that these examples are merely illustrative of the present disclosure, and various changes and modifications may be made within the scope and technical spirit of the present disclosure, and that such variations and modifications fall within the scope of the appended claims.

### Example 1

An exemplary formulation of the aqueous fullerene composition provided herein is as follows.

| Component | Amount | |
|---|---|---|
| Surfactant - TWEEN 80 (polysorbate 80) | 1000 | mg/L |
| Flavor - Benzaldehyde | 1.5 | mg/L |
| Sweetener - Stevia | 5 to 100 | mg/L |
| Fullerene C60 | 80 | mg/L |
| Mineral Ca | 586 | mg/L |
| Mineral Mg | 325 | mg/L |
| Mineral Fe | 5 | mg/L |
| Mineral Zn | 5 | mg/L |
| Mineral Se | 0.01 | mg/L |
| Mineral Cu | 0.2 | mg/L |
| Hydrogen (H₂) | 20 | ppm |

| | |
|---|---|
| Structured Water | Balance |

### Example 2

Another exemplary formulation of the aqueous fullerene composition provided herein is as follows.

| Component | Amount | |
|---|---|---|
| Surfactant - TWEEN 80 (polysorbate 80) | 1000 | mg/L |
| Flavor - Benzaldehyde | 1.5 | mg/L |
| Sweetener - Stevia | 5 to 100 | mg/L |
| Fullerene C60 | 20 | mg/L |
| Mineral Ca | 586 | mg/L |
| Mineral Mg | 325 | mg/L |
| Mineral Fe | 5 | mg/L |
| Mineral Zn | 5 | mg/L |
| Mineral Se | 0.01 | mg/L |
| Mineral Cu | 0.2 | mg/L |
| Hydrogen (H₂) | 20 | ppm |

| | |
|---|---|
| Structured Water | Balance |

### Example 3

An exemplary formulation of the aqueous fullerene composition provided herein is as follows.

| Component | Amount | |
|---|---|---|
| Surfactant - TWEEN 80 (polysorbate 80) | 1000 | mg/L |
| Flavor - Benzaldehyde | 1.5 | mg/L |
| Sweetener - Stevia | 5 to 100 | mg/L |
| C60 Fullerenol | 800 | mg/L |
| Mineral Ca | 586 | mg/L |
| Mineral Mg | 325 | mg/L |
| Mineral Fe | 5 | mg/L |
| Mineral Zn | 5 | mg/L |
| Mineral Se | 0.01 | mg/L |
| Mineral Cu | 0.2 | mg/L |
| Hydrogen (H₂) | 20 | ppm |

| | |
|---|---|
| Structured Water | Balance |

Structured water was prepared as described herein, resulting in a structured water containing the minerals shown in the formulation above at concentrations so that when the other components shown in the formulation above are added, the final concentration of the minerals was as shown. The fullerene and benzaldehyde were added to a mixing vessel capable of generating a vortex when a mechanical stirrer was activated to rotate a mixing blade. The surfactant was added to the mixing vessel along with an amount of structured water that was about 25% of the total weight of the composition. The mechanical stirrer was activated and the speed increased to create a large amount of non-laminar vortices in the water, which generated small vapor bubbles in the water, resulting in cavitation and implosion processes. The product of this process was structured water with water-soluble fullerene with micelles surrounding at least a portion if not all of the fullerene nanoparticles. The remainder of the structured water of the formulation was then added to the vessel, the mechanical stirrer was activated and the speed increased to create a large amount of non-laminar vortices in the water, which generated small vapor bubbles in the water, resulting in cavitation and implosion processes. The product of this process was then output to a mixing device connected to a hydrogen regulated cylinder.

The amount of dissolved hydrogen in the formulation was measured, and additional hydrogen was added if needed to bring the final concentration of hydrogen in the formulation to be 20 ppm.

### Example 4

Another exemplary formulation of the aqueous fullerene composition provided herein is as follows.

| Component | Amount | |
|---|---|---|
| Surfactant - TWEEN 80 (polysorbate 80) | 1000 | mg/L |
| Flavor - Benzaldehyde | 1.5 | mg/L |
| Sweetener - Stevia | 5 to 100 | mg/L |
| C60 Fullerenol | 200 | mg/L |
| Mineral Ca | 586 | mg/L |
| Mineral Mg | 325 | mg/L |
| Mineral Fe | 5 | mg/L |
| Mineral Zn | 5 | mg/L |
| Mineral Se | 0.01 | mg/L |
| Mineral Cu | 0.2 | mg/L |
| Hydrogen (H₂) | 20 | ppm |

| | |
|---|---|
| Structured Water | Balance |

The formulation was prepared as described in Example 3, except that the amount of C60 fullerenol was reduced.

### Example 5

An observational case study was performed in Colombia administering the composition provided herein to cancer patients, the majority of which were in stage 4. The patients had different pathologies and included children (two, 9 and 10 years old) and 10 adults (ages from 39 to 80 years old). Some of the patients also were being treated with chemotherapy in conjunction with the composition provided herein. The diagnosed cancer for each patient is shown in Table 2 below.

TABLE 2

| Patient | Gender | Age | Diagnosis | Chemo |
|---|---|---|---|---|
| 1 | Female | 41 | Infiltrating Ductal Carcinoma Breast | yes |
| | | | In Situ G2 T2 | |
| 2 | Male | 9 | Medulloblastoma | no |
| | (child) | | | |
| 3 | Female | 43 | Left breast cancer stage 3 | yes |
| 4 | Male | 70 | Chronic lymphocytic leukemia | no |
| 5 | Male | 73 | G2 moderately differentiated | Yes |
| | | | adenocarcinoma of the rectum | |
| 6 | Female | 57 | G3 Multicentric Breast Infiltrating | No |
| | | | Ductal Carcinoma | |
| 7 | Male | 72 | Malignant tumor of the rectum | yes |
| 8 | Female | 80 | Adenocarcinoma in the Liver and | Yes |
| | | | Pancreatic Mass | |
| 9 | Male | 9 | Cerebellar tumor | No |
| | (child) | | | |
| 10 | Female | 39 | Carcinoma in the infiltrating and | Yes |
| | | | ulcerated gastric body in stage IV | |
| | | | due to carcinomatosis | |
| 11 | Male | 44 | Original pancreatic tumor in the liver | Yes |
| 12 | Male | 60 | Colon cancer adenocarcinoma | Yes |

The decision whether or not to have chemotherapy was either the patient's own decision or a clinical decision.

The dosing schedule used was as follows. For the adult patients, a 100 mL dose of the formulation shown in Example 3 (delivering an amount of 80 mg fullerenol and between 10-50 ppm H₂) was administered orally on day 1. On days 2 through 6, a 100 mL dose of the formulation shown in Example 4 (delivering an among of 20 mg fullerenol and between 10-50 ppm H₂) was administered orally. On day 7, no formulation was administered. The second and subsequent weeks, the same cycle and dosing schedule began again. The study was carried out for a period of 8 weeks. For the child patients, a 100 mL dose of the formulation shown in Example 4 (delivering an amount of 20 mg fullerenol and between 10-50 ppm H₂) was administered orally on day 1. On days 2 through 6, a 100 mL dose of the formulation shown in Example 4 (delivering an among of 20 mg fullerenol and between 10-50 ppm H₂) was administered orally. On day 7, no formulation was administered. The second and subsequent weeks, the same cycle and dosing schedule began again. The study was carried out for a period of 8 weeks.

The patients were monitored 3 times a week to identify changes in the patient's perception of well-being based on the identification of previous symptoms and ailments.

Each patient was visited every 15 days and asked about personal eating habits, intake of meat, sugar, soft drinks, liquor, drugs, cigarettes. and their family environment, quality of housing, and employment situation, among other aspects. A EuroQ health and well-being survey is being prepared.

In Patient 1, according to a medical report, it was found that the size of the tumor has decreased. In a medical examination, the tumor was almost not perceived by touch. Patient 1 reported that prior to taking the composition provided herein, she felt discouragement and tiredness, and took a long time to recover from the chemotherapy treatments. After participating in the study, Patient 1 reported that she does not feel tired, and that she recovers quickly from the chemotherapy. Patient 1 reported that vomiting after chemotherapy decreased noticeably. Patient 1 reported that she feels vital, sleeps well, eats well, and now can carry out her activities normally. Patient 1 reported no weight loss, and having a high energy and well-being level.

In Patient 2, due to the location of the tumor, it was impossible to do surgery or chemotherapy. Currently, the chances of surviving this type of tumor in children is 5%. The cancer was found by evidence of difficulty in vision called diplopia.

After a month of treatment with the composition provided herein, according to MRI analyses, a decrease in the size of the tumor was observed. In addition, the boy regained his vision.

The pediatrician confirmed the decrease in the size of the tumor, and allowed Patient 2 to resume activities such as traveling, playing soccer, and dancing, which were contraindicated prior to treatment with the composition provided herein. Prior to treatment with the composition provided herein, Patient 2 reported discouragement and tiredness. Patient 2 reported that since treatment with the composition provided herein began, he began feeling better, and that after 8 weeks of treatment he sleeps well, eats well, has his appetite back, does not feel tired, and can play with other children and carry out his activities normally. There was no reported weight loss in Patient 2 during treatment.

Patient 3 was suffering from breast cancer with bone metastasis. Prior to treatment with the composition provided herein, Patient 3 reported taking a long time to recover from chemotherapy, and being tired when doing efforts such as cleaning the home. After 4 weeks of treatment, Patient 3 reported a noticeable reduction in the time for recovery from chemotherapy, and that vomiting after chemotherapy decreased noticeably while taking the composition provided herein. Patient 3 reported that since the treatment with the composition provided herein, she does not feel tired and has felt in good health. There was no reported weight loss in Patient 3 during treatment.

Patient 4 was suffering from chronic lymphocytic leukemia. Prior to treatment, Patient 4 reported feeling lethargic and having a low energy level. After 8 weeks of treatment, Patient 4 reported high energy and an increased level of well-being. He reported that he sleeps better, and has begun to resume normal activities without feeling tired. Patient 4 also reporting an increased appetite, and had no weight loss.

Patient 5 was suffering from adenocarcinoma of the rectum. Patient 5 reported that prior to treatment with the composition provided herein, he could not pass feces on his own and required a colostomy bag. He also took long to recover from chemotherapy and had constant bouts of vomiting after chemotherapy. Patient 5 reports that since treatment with the composition provided herein, he recovers quickly from chemotherapy and vomiting after chemotherapy decreased noticeably. Patient 5 also reported that since treatment with the composition provided herein, he can pass feces normally in the bathroom with needing a colostomy bag. Patient 5 also reported having more energy, feeling vital, and eating and sleeping well. Patient 5 also reported being able to resume normal routine activities. There has no reported weight loss in Patient 5 during the treatment.

Patient 6 was suffering from breast cancer, and reported discouragement and tiredness since diagnosis. Patient 6 was not sleeping well and had a general sense of malaise. Patient 6 reported that since treatment, she sleeps well, eats well, and has not lost weight. Patient 6 reported that she has a higher energy level and a better sense of well-being, and has been able to resume her daily activities.

Patient 7 was undergoing chemotherapy for the treatment of a malignant tumor of the rectum. Patient 7 reported discouragement and tiredness during chemotherapy alone. After treatment with the composition provided herein, Patient 7 reported that he recovered from chemotherapy faster and that there was a noticeable decrease in vomiting after chemotherapy. Patient 7 reported sleeping better, and having a better appetite since treatment with the composition provided herein. Patient 7 reported that he has been able to resume his activities normally and has a better sense of well-being and a higher energy level.

Patient 8 had to withdraw from treatment after three weeks due to an infection unrelated to treatment with the composition provided herein. Prior to withdrawing, Patient 8 reported having higher energy levels and that the treatment was improving her sleep.

Patient 9 is a child suffering from a cerebellar tumor. Facial paralysis and neurogenic bladder presented after surgery. The neurogenic bladder required the use of a catheter to evacuate urine. Following treatment with the composition provided herein, the facial paralysis has decreased, and a catheter to evacuate urine is no longer necessary because Patient 9 feels the need to urinate (3 years after surgery). Patient 9 had suffered from dyslexia, but following treatment with the composition provided herein, the dyslexia has been removed and he writes correctly. After treatment, Patient 9 reported a very positive attitude including at school, a high energy level, and has been able to participate in normal activities at school. Patient 9 reported sleeping well and eating well since treatment, and that cramps he experienced previous to treatment have gone away 100%. Patient 9 reported starting lighting weights and has returned to his taekwondo classes.

Patient 10 was being treated with chemotherapy for stage IV carcinomatosis. Before treatment with the composition provided herein, Patient 10 reported discouragement and tiredness, and frequent vomiting following chemotherapy. On the first day of treatment with the composition provided herein, Patient 10 weighed 43 kilograms, and at the end of a month of treatment with the composition provided herein she weighed 49 kilograms, gaining 6 kilograms in a month. After treatment, she reports that she does not feel tired, that she does not have vomiting after chemotherapy. Patient 10 is working and fully carrying out all her daily activities normally. Patient 10 reported that since treatment she feels vital, sleeps well, eats well, and has high energy and a sense of well-being.

Patient 11 was being treated with chemotherapy for pancreatic tumor in the liver. Liver and pancreatic cancer both very aggressive cancers. Patient 11 has a good attitude although he is very afraid of dying and sometimes has panic attacks. Patient 11 reported vomiting and fever following chemotherapy. After 4 weeks of treatment with the composition provided herein, Patient 11 reported that his vomiting had decreased by 80%, and the fever episodes decreased from a daily frequency lasting 5 hours to sporadic and lasting no more than 1.5 hours. Contrasted computed tomography of the abdomen and pelvis revealed that after 4 weeks of treatment, the size of the tumor decreased and the metastasis was not increasing in size or number. When Patient 11 received the news, he reported feeling hope and from then on he has had a better attitude. Patient 11 reported that since treatment he has been sleeping well and feels good, having more energy.

Patient 12 was being treated with chemotherapy for colon cancer adenocarcinoma. Patient 12 reported that the chemotherapy caused frequent vomiting and tiredness, and she felt very discouraged. After three weeks of treatment with the composition provided herein, Patient 12 reported that vomiting after chemotherapy decreased noticeably. Patient 12 reported that since treatment began, he feels full of energy despite the chemotherapy, and recovers faster from the chemotherapy. He reported eating and sleeping better since treatment, and has a high well-being level.

The results show that the composition provided herein is efficacious and well tolerated in patients, including in patients concurrently undergoing chemotherapy. The study shows that the compositions provided herein significantly improves the outcomes for cancer patients. Combining the compositions provided herein containing fullerene nanoparticles with other therapeutic modalities, such as chemotherapy, radiotherapy and immunotherapy, could synergistically improve treatment efficacy and overcome treatment resistance. The compositions provided herein can improve the efficacy and/or reduce side effects of administration of chemotherapeutic treatment of cancer. The compositions provided herein can improve the quality of life of a cancer patient by improving energy levels, and improving sleep and appetite. The compositions provided herein demonstrated no negative impact on weight (weight loss) and in at least one patient resulted in significant weight gain, reversing wasting. The compositions provided herein demonstrated reduction in tumor size, and arrest of spread to surrounding tissue.

1. An aqueous composition, comprising:
a fullerene;
a micelle-forming agent;
structured water comprising multiple water molecules in a planar orientation where adjacent water molecules are joined by hydrogen bridges forming hexagonal rings of water molecules forming a plane of a two-dimensionally ordered hexagonal matrix arrangement of water molecules, which is replicated in a plurality of planes stacked in a direction perpendicular to the plane of the of two-dimensionally ordered hexagonal matrix arrangement and connected via hydrogen bridges to form multiple layers of the two-dimensionally ordered hexagonal matrix arrangement, forming a plurality of three-dimensional helical cage structures of polygonal water molecules, wherein each of the helical cage structures has a central hollow lumen;
dissolved hydrogen gas; and
minerals.

2. The aqueous composition of claim 1, wherein:
the fullerene comprises an unmodified C60 or C70 fullerene;
the fullerene comprises a C60 or C70 fullerene modified to include one or more than one hydrophilic group on a surface of the fullerene, wherein the hydrophilic group is a hydroxyl group, a carboxy group, a carboxylic acid group, an alcohol group, an amine group, or a sulfonate group; or
the fullerene comprises a C60 or C70 fullerene modified to be complexed with a water-soluble polymer selected from the group consisting of a polyethylene glycol, a dextran, a polyvinyl alcohol, a polysaccharide, and a protein: or
the fullerene comprises fullerenol.

3. (canceled)

4. (canceled)

5. (canceled)

6. (canceled)

7. The aqueous composition of claim 1, wherein;
the amount of the fullerene is 1 mg/L to 30,000 mg/L;
the amount of the micelle-forming agent is 50 mg/L to 800 g/L;
the amount of hydrogen gas present is in a range of 1 to 50 ppm; and
the minerals are present in an amount from 0.005 mg/L to 800 mg/L.

8. The aqueous composition of claim 1, wherein the micelle-forming agent comprises a surfactant, a liposoluble agent, an oil, or a combination thereof.

9. (canceled)

10. The aqueous composition of claim 1, wherein the micelle-forming agent comprises a nonionic surfactant selected from the group consisting of a sorbitan ester, a polyalkoxylated sorbitan fatty acid ester, a polyoxyethylene ether, a polysorbate, and a combination thereof.

11. (canceled)

12. (canceled)

13. (canceled)

14. (canceled)

15. (canceled)

16. (canceled)

17. The aqueous composition of claim 1, further comprising a sweetener, a flavoring agent, a flavor enhancer, or a combination thereof, wherein the sweetener and/or the flavoring agent and/or the flavor enhancer, when present, is present in an amount from about 0.0001 to 20 wt %.

18. The aqueous composition of claim 17, wherein:
(a) the sweetener is selected from the group consisting of sucrose, stevia, steviol glycosides, monk fruit extract, sucralose, isomalt, aspartame, saccharin, acesulfame potassium, neotame, advantame, lactitol, xylitol, high-fructose corn syrup, and combinations thereof; or
(b) the flavor enhancer is selected from among a citric acid, a lactic acid, a sodium gluconate, a sodium gluconolactate, an ethyl lactate, a sodium lactate, a sodium acid sulfate, malic acid, and combinations thereof: or
(c) both (a) and (b).

19. (canceled)

20. (canceled)

21. (canceled)

22. (canceled)

23. (canceled)

24. (canceled)

25. (canceled)

26. (canceled)

27. A method of administering photodynamic therapy to a subject in need thereof, the method comprising:
selecting a site of the subject where the photodynamic therapy is to be administered;
administering to the subject an aqueous composition of claim 1, which can be administered alone or in combination with a pharmaceutically acceptable carrier; and
exposing the site to natural or artificial light for a period of time effective to achieve a therapeutic effect.

28. The method of claim 27, wherein the subject has a disorder selected from among a cancer, psoriasis, acne, actinic keratosis, eczema, seborrhea and hyperkeratinosis.

29. The method of claim 28, wherein the cancer is selected from among breast, prostate, liver, colorectal, gastrointestinal, pancreatic, skin, thyroid, cervical, lymphoid, hematopoietic, bladder, lung, renal, ovarian, uterine, and head or neck cancer.

30. The method of any claim 27, wherein the administering is nonparenteral.

31. (canceled)

32. A method of treating cancer in a subject, comprising administering to the subject a therapeutically effective amount of an aqueous composition of any one of claims 1 to 26, which can be administered alone or in combination with a pharmaceutically acceptable carrier,
wherein the administering is for a period of 2 weeks to 12 months, and
wherein a daily dosage of fullerene in the composition is in the range of 20 mg to 80 mg.

33. The method of claim 32, wherein:
the cancer is selected from among breast, prostate, liver, colorectal, gastrointestinal, pancreatic, skin, thyroid, cervical, lymphoid, hematopoietic, bladder, lung, renal, ovarian, uterine, and head or neck cancer, or
the cancer is selected from among an adenocarcinoma, a medulloblastoma, chronic lymphocytic leukemia, and a cerebellar tumor.

34. (canceled)

35. The method of claim 32, wherein the aqueous composition is administered alone or in combination with a second therapeutic treatment, wherein the second therapeutic treatment comprises a chemotherapy, a radiotherapy, an immunotherapy, or a combination thereof.

36. (canceled)

37. The method of claim 32, wherein the administering includes daily administration, twice weekly administration, or weekly administration.

38. The method of claim 37, wherein the administering includes a drug holiday of one or more consecutive days during with the composition is not administered.

39. The method of claim 37, wherein the administering includes administering the composition on a dosing schedule that includes administering:
(a) daily for a period of 6 days followed by one day of no treatment, and repeating for 2 to 52 cycles of the schedule; or
(b) daily for a period of 5 days followed by two days of no treatment, and repeating for 2 to 52 cycles of the schedule; or
(c) daily for 4 days followed by one day of no treatment, and repeating for 2 to 73 cycles of the schedule; or
(d) daily for 3 days followed by one day of no treatment, and repeating for 2 to 91 cycles of the schedule; or
(e) daily for 2 days followed by one day of no treatment, and repeating for 2 to 122 cycles of the schedule; or
(f) every other day for a period of 2 to 52 weeks.

40. (canceled)

41. (canceled)

42. The method of claim 39, wherein the administering includes providing a boost dose comprising a dosage of fullerene that is 10% to 400% higher than the daily dosage of fullerene.

43. The method of claim 42, wherein the boost dose is administered at the beginning of each dosing schedule, or the end of each dosing schedule, or after 2, 3, 4, or more cycles of the dosing schedule.

44. (canceled)
